SEQUENCE LISTING (40 sequences)

SEQ ID NO: 1 - protein, 11 aa, Synechocystis PCC6803
MAEAFIAENTA

SEQ ID NO: 2 - protein, 12 aa, Synechocystis PCC6803
QVGLALERSDLL

SEQ ID NO: 3 - protein, 12 aa, Fremyella diplosiphon
GRLEEVVAARTA

SEQ ID NO: 4 - protein, 11 aa, Synechocystis PCC6803
MATTVQLSDQS

SEQ ID NO: 5 - protein, 11 aa, Synechocystis PCC6803
VVFSNISAGED

SEQ ID NO: 6 - protein, 12 aa, Fremyella diplosiphon
GRLEEVVAARTA

SEQ ID NO: 7 - protein, 247 aa, Fremyella diplosiphon
MLKSMEASLRNRQHPLIRKLADSIEEIWQKYLDISPYSVPEGLGYVEGHLEGERLIIENHCYQAPQFRKLHLELAQVGNGLDILHCVMFPNPEYPIPMFGTDLVGGKGGLISAAIADLSPISSDRSLPSRYYQQLSALPEIEFSEPRALPPWGDIFSHFCLFVRPENAEEEEKFHNRVREYLTIHCQIAASETPLTSNLDISRVLAGQRNYCTKQQQNDKTRRVLEKSFGKEWADRYMTTMLFDYVA

SEQ ID NO: 8 - protein, 245 aa, Anabaena PCC7120
MSLTSIPSLREQQHPLIRQLADCIEEVWHQHLDLSPYHLPAELGYVEGRLEGEKLTIENRCYQTPQFRKMHLELAKVGNMLDILHCVMFPRPEYDLPMFGCDLVGGRGQISAAIADLSPVHLDRTLPESYNSALTSLNTLNFSQPRELPEWGNIFSDFCIFVRPSSPEEEAMFLGRVREFLQVHCQGAIAASPVSAEQKQQILAGQHNYCSKQQQNDKTRRVLEKAFGVDWAENYMTTVLFDLPE

SEQ ID NO: 9 - protein, 248 aa, Synechocystis PCC6803
MAVTDLSLTNSSLMPTLNPMIQQLALAIAASWQSLPLKPYQLPEDLGYVEGRLEGEKLVIENRCYQTPQFRKMHLELAKVGKGLDILHCVMFPEPLYGLPLFGCDIVAGPGGVSAAIADLSPTQSDRQLPAAYQKSLAELGQPEFEQQRELPPWGEIFSEYCLFIRPSNVTEEERFVQRVVDFLQIHCHQSIVAEPLSEAQTLEHRQGQIHYCQQQQKNDKTRRVLEKAFGEAWAERYNSQVLFDVIQ

SEQ ID NO: 10 - DNA, 35 bp, Fremyella diplosiphon
aaggtaggttaatcctatactcaaagagaagcgta

SEQ ID NO: 11 - DNA, 36 bp, Fremyella diplosiphon
aaacggattgctttaatcaatttcaggttcaaaaaa

SEQ ID NO: 12 - DNA, 18 bp, Fremyella diplosiphon
  misc_feature (8)..(11): n is a, c, g, or t
ttgcacannnnttgcaca

SEQ ID NO: 13 - DNA, 13 bp, Fremyella diplosiphon
aaatttgcacaaa

SEQ ID NO: 14 - DNA, 67 bp, Fremyella diplosiphon
tatgatgaaatgagagaattttgcactcaatttgcacaactatttgtaatgggcatgggaaattggg

SEQ ID NO: 15 - DNA, 72 bp, Fremyella diplosiphon
atttaaatttgcacaaatttgcacaaacggattgctttaatcaatttcaggttcaaaaaacagctgaacgct

SEQ ID NO: 16 - DNA, 73 bp, Fremyella diplosiphon
tctgcaaattgcacagatattgcacaggctatacgccaaggtaggttaatcctatactcaaagagaagctatg

SEQ ID NO: 17 - DNA, 73 bp, Pseudanabaena PCC7409
tctatgctttgcacaaaaactgcacaaattagttgttttaatctttttaatcccaaagtcgctaagccaaaat

SEQ ID NO: 18 - DNA, 35 bp, Fremyella diplosiphon
aattacgaattacgaattacgaattacgaattacg

SEQ ID NO: 19 - DNA, 65 bp, Fremyella diplosiphon
cgtgaaattcataacttaattacgaattagtaactacgaattacgaattatttatgagttattta

SEQ ID NO: 20 - DNA, 66 bp, Fremyella diplosiphon
ttgtttgtactaattttaattacgaattacgaattacgaattactttctcaagctgtctttatttt

SEQ ID NO: 21 - DNA, 65 bp, Fremyella diplosiphon
atccctatccgtaacgtaattacgaattagtaactacgaattacgagttgttaatactaatcggt

SEQ ID NO: 22 - DNA, 65 bp, Fremyella diplosiphon
actcaaaccctcagcttaattacgaattacgaattacgaattacgaattagtaatttaggttgag

SEQ ID NO: 23 - DNA, 28 bp, Fremyella diplosiphon
gcaaattgcacagatattgcacaggcta

SEQ ID NO: 24 - DNA, 28 bp, Artificial Sequence
  substitution mutation within pcyA wild type L Box, orignated from Fremyella diplosiphon
gcaaatacgtacgataagtctacggcta

SEQ ID NO: 25 - DNA, 39 bp, Fremyella diplosiphon
taacttaattacgaattagtaactacgaattacgaatta

SEQ ID NO: 26 - DNA, 39 bp, Artificial Sequence
  substitution mutation within STRR5 repeat sequence, orginated from Fremyella diplosiphon
taactttggatacatgcatctcgagatgcatgtatccaa

SEQ ID NO: 27 - DNA, 23 bp, Fremyella diplosiphon
tgytaycarachcchcarttycg

SEQ ID NO: 28 - DNA, 23 bp, Fremyella diplosiphon
cgdgtyttrtcrttytgytgytg

SEQ ID NO: 29 - DNA, 23 bp, Fremyella diplosiphon
gtttagatattctgcactgtgtg

SEQ ID NO: 30 - DNA, 23 bp, Fremyella diplosiphon
ttttgtacagtaattgcgctgtc

SEQ ID NO: 31 - DNA, 31 bp, Artificial Sequence
  primer FdpcyAEcoRIfwd, originated from Fremyella diplosiphon, with EcoRI restrictive site added.
ggaattcatcttgaagtctatggaagcgtcg

SEQ ID NO: 32 - DNA, 35 bp, Artificial Sequence
  primer FdpcyAHindIIIrev, orginated from Fremyella diplosiphon, with restriction site HindIII added.
cccaagcttttaagctacatagtcaaaaagcatag

SEQ ID NO: 33 - DNA, 19 bp, Fremyella diplosiphon
gacgaggcttgcggagtgg

SEQ ID NO: 34 - DNA, 23 bp, Fremyella diplosiphon
tctagatgcagtttgcgaaattg

SEQ ID NO: 35 - DNA, 22 bp, Fremyella diplosiphon
gcccacaagcctagattaagaa

SEQ ID NO: 36 - DNA, 31 bp, Artificial Sequence
  primer pcyAGUS1, originated from Fremyella diplosiphon, with restrictive endonulease added.
gcggcatgcacaaaccacagaaatgcctgag

SEQ ID NO: 37 - DNA, 51 bp, Artificial Sequence
  primer newpcyAGUS2, originated from Fremyella diplosiphon, with restriction endonuclease site added
gcgggatcccatagacttcaacatacgcttctctttgagtataggattaac

SEQ ID NO: 38 - DNA, 28 bp, Artificial Sequence
  primer for PCR, orgination unknown
gcgctcgagatgcatgtatccaaagtta

SEQ ID NO: 39 - DNA, 28 bp, Artificial Sequence
  primer for PCR, origination unknown
gcgctcgagatgcatgtatccaatttat

SEQ ID NO: 40 - DNA, 97 bp, Artificial Sequence
  primer for PCR purpose. Originated from Fremyella. Diplosiphon, restriction site added and L Box mutation included.
gcgggatcccatagacttcaacatacgcttctctttgagtataggattaacctaccttggcgtatagccgtagacttatcgtacgtatttgcagatt

(19) United States
(12) Patent Application Publication
(10) Pub. No.: US 2010/0093051 A1
(43) Pub. Date: Apr. 15, 2010

(54) LIGHT REGULATED TRANSCRIPTION SYSTEM FOR USE IN PROKARYOTIC ORGANISMS

(76) Inventors: David M. Kehoe, Bloomington, IN (US); Lina Li, Bloomington, IN (US); Richard M. Alvey, Bloomington, IN (US)

Correspondence Address: BOSE MCKINNEY & EVANS LLP, 111 MONUMENT CIRCLE, SUITE 2700, INDIANAPOLIS, IN, 46204, US

(21) Appl. No.: 12/446,788

(22) PCT Filed: Oct. 26, 2007
(86) PCT No.: PCT/US07/82622

§ 371 Date: Nov. 18, 2009

Related U.S. Application Data
(60) Provisional application No. 60/863,230, filed on Oct. 27, 2006.

(51) Int. Cl. C12N 13/00

(52) U.S. Cl. 435/173.1

(57) ABSTRACT

A process for regulating transcriptional activity of a gene is provided. The process comprises introducing genetic material into an organism, placing a gene downstream of a light-regulated promoter within the organism having a light responsive element and subjecting the organism to an exogenous light source. The genetic material is selected from a photosynthetic bacterium and the light source is configured to control the level of transcriptional activity of the gene.

### RELATED APPLICATIONS

This application claims priority to U.S. Provisional Patent Application Ser. No. 60/863,230, filed Oct. 27, 2006, the disclosure of which is expressly incorporated by reference herein.

This invention was made in part with government support under grant reference number MCB-0519433 awarded by the National Science Foundation. The Government has or may have certain rights in the invention.

### TECHNICAL FIELD

The present invention generally relates to a system for regulating the transcriptional activity of target genes of prokaryotic organisms by applying an exogenous light source of a specific wavelength (color). The transcriptional activity is controlled by placing a target gene downstream of a light-color-regulated cyanobacterial promoter containing a light color responsive element and then selectively activating/deactivating the light source as needed.

### BACKGROUND

While many promoters and transcriptional regulatory systems are known in nature, only a small fraction of these have been adapted for use as molecular biology tools. These may be broadly categorized into two groups, constitutive and inducible promoters. Inducible promoters are useful for many applications in molecular biology and biochemistry, particularly as they have the ability to tightly regulate the timing and genetic expression of proteins.

Many different inducible prokaryotic promoters are known and typically fall into one of two operation modes, positive operation modes and negative operation modes. In a positive operation mode, the genes are off by default and may be turned on by activators. The binding of the activator (regulatory protein) to the regulatory region of the DNA leads to an increase in transcription. In a negative operation mode, the interaction between a repressor protein and a regulatory sequence within the promoter prevents transcription of the gene. The loss of repressor binding leads to increased expression from the gene.

Some of the known types of prokaryotic inducible promoters include: 1) the lac operon system which uses the lac promoter and the lac Repressor protein, whose DNA binding affinity can be artificially decreased by the presence of a small molecule inducer called IPTG; 2) the tetracycline responsive system, which includes the tetracycline repressor protein TetR, a tetracycline operator sequence (tetO), and a tetracycline transactivator fusion protein (tTA), which is the fusion of TetR and a herpes simplex virus protein 16 (VP16); 3) the arabinose responsive system, where the arabinose-inducible araBAD promoter (P_{BAD}) and regulator (AraC) of Escherichia coli (E. coli) can be used to control gene expression; 4) the T7 system, of which the expression of T7 polymerase-recognized promoters is controlled by T7 polymerase levels, which in turn are controlled by lac promoter regulation of T7 gene expression; and 5) the rhamnose responsive system, of which the rhaT promoter is regulated by the abundance of the sugars L-rhamnose and D-glucose. In addition to these systems, there are prokaryotic promoters that are induced by copper, by heat shock and by cold treatment, among others.

Many existing prokaryotic gene regulation systems rely on the addition of chemical elicitors; however, the local concentration of these chemical elicitors is often difficult to control, they take time to reach their targets, and they cannot be removed or reduced in concentration once applied. Moreover, many of these systems retain some level of transcriptional activity from the inducible promoter, even when the system is under a non-inductive condition. Thus, systems that have been found to be highly inducible, such as the lac operon system, cannot be completely shut off under non-inducing conditions. This is a significant shortcoming when working with genes whose protein products affect cell physiology or are toxic in small amounts. Also, systems that can be completely shut off under non-inducing conditions, such as the arabinose induction system, can only be weakly induced, which often provides insufficient amounts of protein product.

In addition, many chemicals and treatments currently in use as inducing agents dramatically alter the molecular physiology of E. coli. For example, the use of such sugars as lactose, arabinose and rhamnose all have pleiotropic effects on these cells, and treatments such as cold or heat shock, or exposure to high concentrations of metals such as copper, also alter the physiology of these cells. Many of these systems also display an “on-off” response in E. coli due to forward feedback mechanisms in place within the systems. Thus, variations in induction levels occur as a result of changes in the percentage of cells that are activated within a population rather than a gradation of expression within a single cell. The present invention is intended to overcome and improve upon these and other shortcomings of the prior art.

### SUMMARY OF THE INVENTION

The present teachings provide a system for regulating transcriptional activity in prokaryotic organisms, such as E. coli. The activation and inactivation of the transcriptional activity is regulated by the color of ambient light that is provided to the cell culture. Exemplary light sources according to the present teachings include, for instance, red and green lights. blue and green lights or red and far red lights, particularly as these regions of the light spectrum are effective for activating and inactivating gene expression from a single regulated promoter. It should be understood and appreciated herein, however, that other exemplary light sources may also be useful in accordance with the present teachings. For example, in other exemplary embodiments, a series of light responsive systems may be used wherein each of the systems respond to different light colors to thereby serially activate and/or inactivate a whole series of genes encoding, for example, a series of enzymes that act consecutively within a biosynthetic pathway. Moreover, it should also be understood and appreciated that the various light absorbing photoreceptors disclosed herein may work through various types of two component regulatory systems, such as for instance, a system wherein two proteins are in the pathway (i.e., a photoreceptor and a response regulator that is also the transcription factor of the system). In each of these exemplary teachings, light is an efficient stimulus for activating and deactivating gene expression because it can be rapidly and uniformly applied, in very specific doses, to growing cell cultures and can be instantaneously and completely removed at will.

In one specific embodiment of the present teachings, the transcription system operates in E. coli and consists of one of three cyanobacterial photoreceptors (either RcaE or one of the chimaeric molecules PixJ1:RcaE or Cph1:RcaE) and two additional cyanobacterial signal transduction proteins, one of which is the transcription factor RcaC. This system is capable of activating or inactivating the transcription of a target gene in response to exogenously applied light, either red and green (for RcaE), blue and green (for PixJ1:RcaE) or red and far red (for Cph1:RcaE). The control of target gene transcription is achieved by its placement downstream of a light-regulated cyanobacterial promoter, cpc2, which contains a cis-acting, light responsive activating element called the L Box that is bound by RcaC.

Unlike many conventional gene regulation systems, the present teachings do not require the use of abiotic conditions or chemical elicitors that perturb normal cellular processes, as well as do not involve transcriptional activity of the inducible promoter when the system is under a non-inductive condition. Moreover, the transcriptional activity of the promoter can be tightly controlled by adjustment of the ratio of either red light to green light, or blue light to green light, or of red light to far red light provided. As such, the promoter operates at a very high level when it is under a fully induced condition. According to this exemplary embodiment, the response can be completely reversed by switching the ambient light color ratio in the opposite direction.

According to another aspect of the present teachings, the expression of a target gene can be deactivated or shut off under non-inducing conditions, while still allowing high levels of gene expression to be enabled under inducing conditions. For instance, according to one exemplary example involving the cyanobacterium Fremyella diplosiphon UTEX 481 (also Tolypothrix sp. PCC 7601), from which this system was derived, over about 30% of the total soluble cellular protein is synthesized from the gene that is driven by the promoter, and this promoter is activated to an extremely high level by this light color activation, and no mRNA from this gene is detectable under non-inductive conditions.

The present teachings also allow the activation and inactivation of a target promoter within a prokaryotic organism by changing the ratio of two wavelengths of light provided exogenously. One exemplary prokaryotic organism according to the present teachings includes E. coli. As the present teachings use either red and green light or blue and green light or red and far red light as the eliciting agent (which is almost not used or sensed by E. coli naturally), it does not have any known significant effect on the cells nor affect the expression of any other promoters within the organism.

The systems of the present teachings are also configured to respond to genetic expression in a rheostat type manner, rather than as an “on-off” response mechanism. More particularly, the present teachings are configured to allow gene expressions to be altered in a gradual fashion within a single cell, and evenly throughout the population. As such, this allows fine control of expression levels of target genes through variation in the ratio of red and green, blue and green, or red and far red light.

According to one exemplary embodiment, a process for regulating the transcriptional activity of a gene is provided. The process comprises introducing genetic material that encodes the components of a light regulated transcriptional control system into a prokaryotic organism, then placing additional genetic material encoding the protein whose synthesis is to be regulated downstream (3′) of a light-regulated promoter having a light responsive element and subjecting the cells that have been transformed with this genetic material to an exogenous light source of a specific wavelength. The genetic material encoding the components of the light controlled regulatory system is selected from a cyanobacterium, the genetic material encoding the protein whose synthesis is to be regulated can come from any organism or can be synthesized de novo, and the light source is configured to control the level of transcriptional activity in the gene.

According to one specific embodiment, a process for regulating transcriptional activity of a gene is provided. The process comprises introducing genetic material into an organism, placing a gene (i.e., a prokaryotic or eukaryotic gene) downstream of a light-regulated promoter within the organism having a light responsive element and subjecting the organism to an exogenous light source. The genetic material is selected from a photosynthetic bacterium and the light source is configured to control the level of transcriptional activity of the gene.

According to another specific embodiment, a process for regulating transcriptional activity of a gene is provided comprising introducing genetic material from a cyanobacterial photoreceptor into a prokaryotic organism; placing a gene in the organism downstream of a cpc2 promoter, the cpc2 promoter having a light responsive element; and subjecting the organism to an exogenous light source, the light source being configured to control the level of transcriptional activity in the prokaryotic gene by adjusting the ratio of light colors provided by the light source. According to this specific embodiment, the cpc2 promoter is obtained from at least one of Synechococcus sp. PCC7335 and Fremyella diplosiphon.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above-mentioned aspects of the present teachings and the manner of obtaining them will become more apparent and the teachings will be better understood by reference to the following description of the embodiments taken in conjunction with the accompanying drawings, wherein:

FIGS. 1a and 1b show a diagram illustrating exemplary components in accordance with the present teachings;

FIG. 2 shows a schematic representation of an exemplary mechanism of action in accordance with the present teachings;

FIG. 3 shows the expression vector pBAD-MycHisC with rcaE, rcaF and rcaC inserted for expression in E. coli in accordance with the present teachings;

FIG. 4 shows the plasmid pDR3′, which contains the two genes that encode the enzymes heme oxygenase and phycocyanobilin oxidoreductase and a lacZ reporter gene (ALPHA) under the transcriptional control of a cpc2 promoter containing the L Box;

FIG. 5 shows exemplary data demonstrating that the activity of the non-light responsive cpc1 promoter (PCc412G), when modified to include an L Box from the red light inducible cpc2 promoter (PCc-cpc2LB), becomes red light inducible;

FIG. 6A shows relative positions and orientations of genes/ORF surrounding pcyA within the genome of F. dilposiphon in accordance with the present teachings;

FIG. 6B shows the complete amino acid sequence of F. diplosiphon (F, dip.) pcyA aligned with pcyA genes from Anabaena sp. PCC 7120 (Ana 7120) and Synechocystis sp. PCC 6803 (Syn 6803) in accordance with the present teachings;

FIG. 7A demonstrates that F. diplosiphon PcyA converts BV to PCB as detected by HPLC in accordance with the present teachings;

FIG. 7B shows the phytochrome difference spectrum of Cph1 incubated with MBP-PcyA bilin reductase assay reaction products in accordance with the present teachings;

FIG. 8 shows Southern blot analysis results used to determine sequences in the F. diplosiphon genome that were closely related to the isolated pcyA gene;

FIG. 9A shows a summary of data collected from three independent RNA blot analyses using a fragment of the F. diplosiphon pcyA gene as a probe in accordance with the present teachings;

FIG. 9B shows an autoradiogram of one of the RNA blots used to obtain the results presented in FIG. 9A after hybridization with a F. diplosiphon pcyA probe;

FIG. 10A shows transcription start sites determined during growth in RL and GL for pcyA using a ribonuclease protection assay;

FIG. 10B shows transcription start sites determined during growth in RL and GL for cpc2 by using a primer extension analysis;

FIG. 11A shows relative positions of potential regulatory elements called STRR5 repeats (“SR5”) and L Boxes (“L”) within the regions of the F. diplosiphon genome near pcyA and cpeCDESTR;

FIG. 11B shows sequence alignments of L Boxes within the light regulated promoters of three F. diplosiphon genes and the cpc2 gene of Pseudanabaena sp. PCC 7409;

11C shows alignment of F. diplosiphon STRR5 repeats sequences in accordance with the present teachings;

FIG. 12A shows relative rates of GUS activity per mg protein in extracts derived from F. diplosiphon cells transformed with indicated plasmids and grown in either RL or GL prior to harvest;

FIG. 12B shows sequence alignments of the pcyA wild type L Box and STRR5 repeat sequences and the sequences used to replace them in ppcyA-LGUS and ppcyA-SGUS; and

FIG. 13 shows a proposed model of how the Rca system regulates CCA controlled genes in F. diplosiphon.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

The present invention is directed to systems for introducing genetic material into prokaryotic organisms to create transcriptional control systems capable of being regulated by exogenously applied light sources. More specifically, the present teachings involve the introduction of several genes of cyanobacterial origin into E. coli, some of which may exist in the chromosome and some of which may exist in a plasmid, in order to create a transcriptional control system that is regulated by light color in that organism. It should be understood and appreciated herein that the genes placed downstream of the light-regulated promoter within the organisms of the present invention may include either prokaryotic genes or eukaryotic genes. As such, the present teachings are not intended to be limited herein.

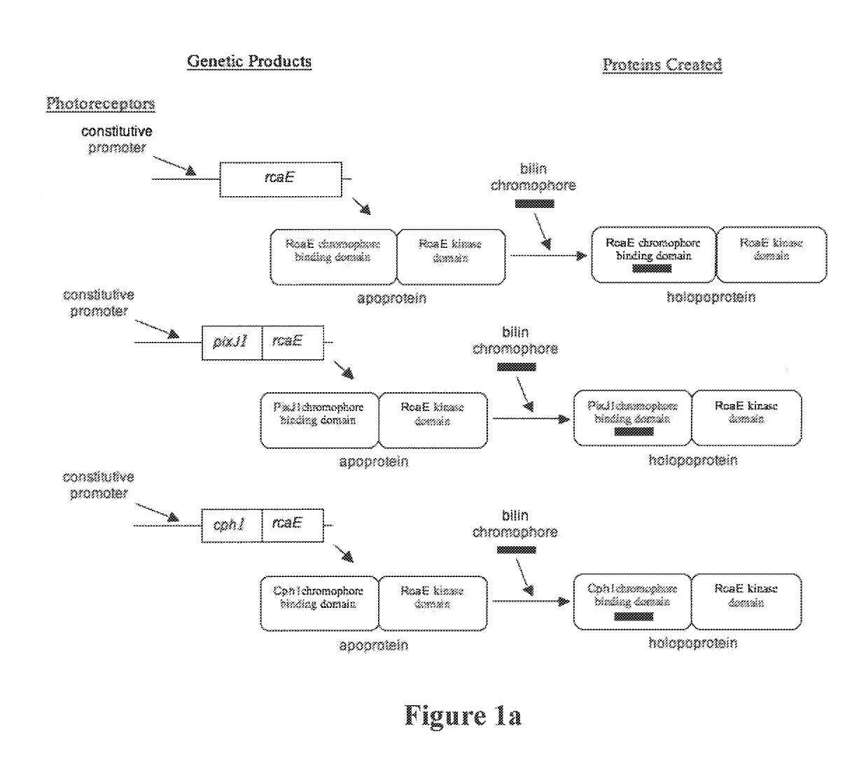

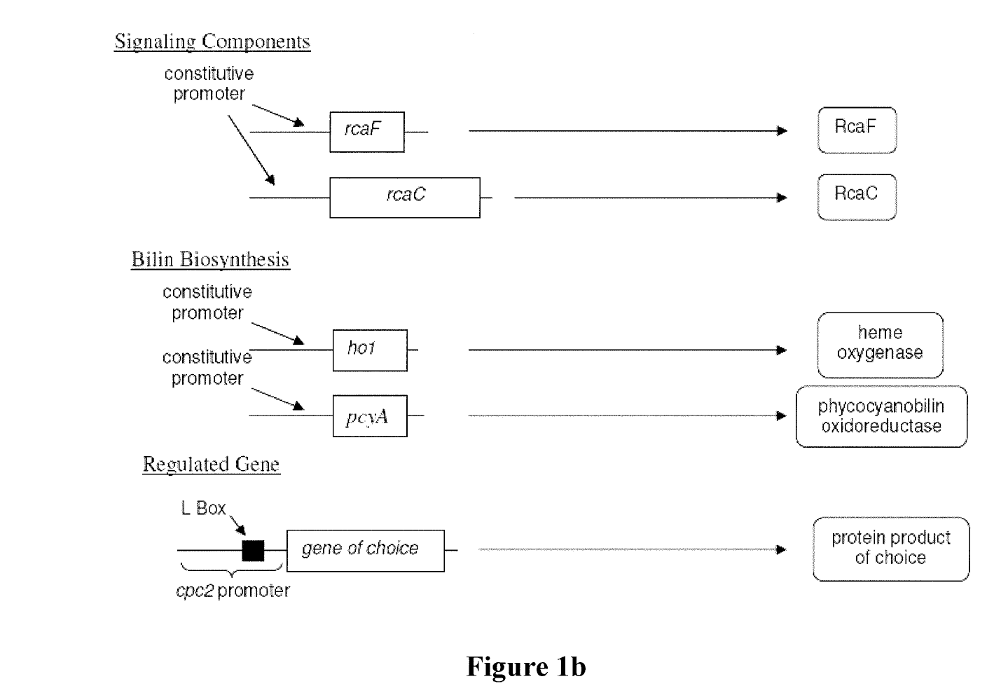

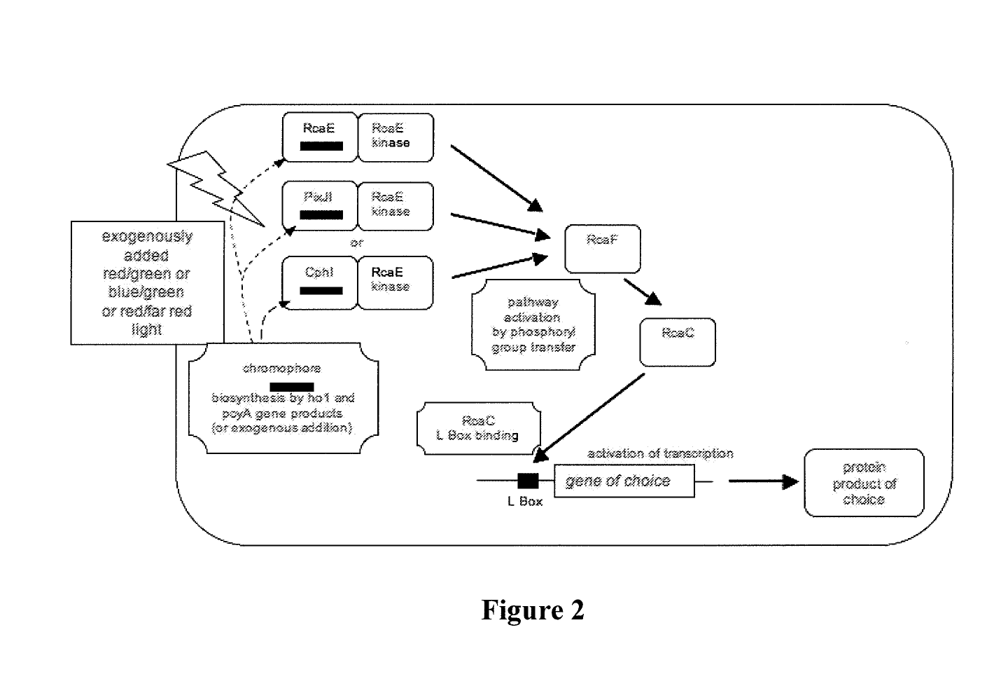

According to one exemplary embodiment, the genetic material introduced into the organism is selected from a photosynthetic bacterium, such as filamentous cyanobacterial species. Exemplary genes of cyanobacterial origin include, for instance, (1) ho1, which encodes heme oxygenase; (2) pcyA, which encodes phycocyanobilin oxidoreductase; (3) pixJ1:rcaE, a chimaeric photoreceptor containing the approximately 500 N-terminal amino acids from PixJ1 fused to approximately 420 amino acids from the C-terminal portion of RcaE; (4) cph1:rcaE, a chimaeric photoreceptor containing the approximately 285 N-terminal amino acids from Cph1 fused to approximately 420 amino acids from the C-terminal portion of RcaE; (5) rcaF, encoding the single domain response regulator RcaF; (6) rcaC, encoding RcaC, which contains four known domains. The chromophores synthesized by ho1 and pcyA could alternatively be supplied exogenously. This system controls the expression of target genes of choice through the placement of these genes immediately 3′ of the cpc2 promoter within a multicopy, autonomously replicating plasmid carrying a selectable marker. The promoter is diametrically regulated by the control system as described herein in a rheostat-like manner, depending on the ratio of either red to green light provided (for cells containing the RcaE photoreceptor), blue to green light provided (for cells containing the PixJ1:RcaE photoreceptor), or red to far red light provided (for cells containing the Cph1:RcaE photoreceptor) from exogenous light sources. This will provide a tightly tunable system for a promoter whose expression level can be reversibly regulated nearly instantaneously by light color. The components of this invention are diagrammed in FIG. 1, and a schematic representation of the mechanism of action of the invention is diagrammed in FIG. 2.

The cpc2 promoter of the present teachings can be obtained from a species capable of light color acclimation, such as the marine cyanobacterium Synechococcus sp. PCC 7335 (https://research.venterinstitute.org/moore/SingleOrganism.do?speciesTag=S7335) or the freshwater cyanobacterium Fremyella diplosiphon (F. diplosiphon UTEX 481 (Calothrix/Tolypothrix PCC 7601)) (Alvey et al. 2006), whereas the pixJ1 and cph1 genes can be obtained from the cyanobacterium Synechocystis sp. PCC 6803 (see http://www.kazusa.or.jp/cyano/Synechocystis/index.html). In F. diplosiphon, the genes are tightly regulated and highly expressed, as the proteins they encode make up over 30% of the total protein in the cell (Bennett and Bogorad 1973). Moreover, in F. diplosiphon, it has been found that a total fluence of 3.000 μmol m⁻² of red light is sufficient to completely activate cpc2 transcription within less than 10 minutes, while a total fluence of 2.500 μmol m⁻² of green light is sufficient to inactivate it. In addition, in vitro results show that two minutes of red light at 120 μmol m⁻² s⁻¹ is sufficient to photoactivate Cph1, while two minutes of far red light at 120 μmol m⁻²s⁻¹ is sufficient to inactivate it. For PixJ1, photoactivation can be achieved in vivo by exposure to blue light for two minutes at 80 μmol m⁻²s⁻¹, and it can be inactivated by green light for two minutes at 30 μmol m⁻²s⁻¹. (see Gambatta and Lagarias 2001; Yoshihara et al. 2006; and Oelmuller, et al. 1988).

The three genes that make up this exemplary regulatory system have been cloned from two different species, Synechococcus sp. PCC7335 and F. diplosiphon, and the encoded proteins RcaE, RcaF and RcaC characterized (Chiang et al. 1992; Kehoe and Grossman 1996; Kehoe and Grossman 1997; Terauchi et al. 2004; Li and Kehoe 2005; Rollo and Kehoe, unpublished data). Although it will be desirable to construct this system by transferring the genes encoding these components from either species directly into E. coli, along with the red light activated and green light inactivated cpc2 promoter, such process may be difficult to perform.

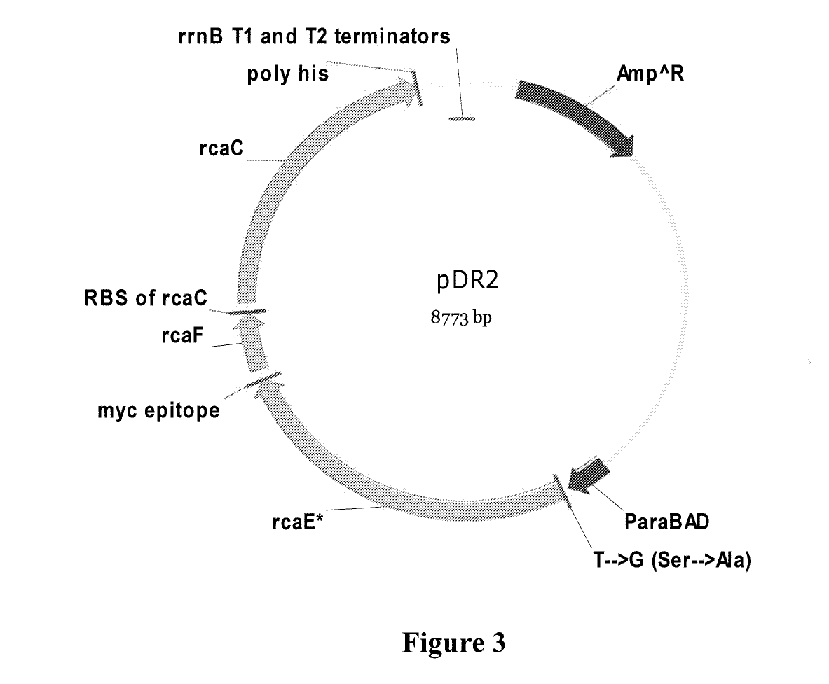

First, the photoreceptor RcaE is an apoprotein that has an open-chain tetrapyrrole (bilin) chromophore covalently attached within a “GAF” domain in the amino terminal portion of this photoreceptor (Terauchi et al. 2004). Prior to introducing RcaE into E. coli, the RcaE may require some modification. More particularly, despite significant effort, the identity of the bilin that attaches to RcaE is unknown (Terauchi et al. 2004). This is important since the type of bilin attached determines the resulting light absorption properties of the RcaE photoreceptor. Also, at least the RcaE photoreceptor from F. diplosiphon is not capable of autocatalytically attaching a bilin in E. coli (Terauchi et al. 2004). It is not yet clear whether the RcaE photoreceptor from Synechococcus sp. PCC7335 is capable of autocatalytic chromophore attachment. Without an attached bilin, at least the F. diplosiphon RcaE photoreceptor cannot act as a photoreceptor in E. coli cells. It has been discovered that this problem may be circumvented, however, through the use of the equivalent RcaE photoreceptor and response regulators RcaF and RcaC from Synechococcus sp. PCC7335. The genes encoding these three proteins are clustered within the Synechococcus sp. PCC7335 genome, which has facilitated their manipulation. These have already been cloned into the expression vector pBAD-MycHisC (Invitrogen) for expression in E. coli (FIG. 3). Alternatively, this problem will be circumvented through the construction of chimeric proteins formed by the fusion of two closely related photoreceptors. This will be facilitated by the fact that the three dimensional structure of the chromophore binding domain of a member of this group of photoreceptors has been solved, thus allowing simple identification of corresponding structural domains (Wagner et al. 2006).

Two exemplary chimaeric proteins according to the present teachings include PixJ1/RcaE and Cph1/RcaE. The region of the pixJ1 gene from Synechocystis sp. PCC 6803 encoding the region containing the chromophore binding domain (starting with the single letter amino acid code MAEAFIAENTA . . . and ending at the sequence . . . QVGLALERSDLL) will be amplified using the polymerase chain reaction (PCR) and covalently attached, using standard molecular biology techniques, to the portion of the rcaE gene from F. diplosiphon encoding the region of the protein beginning at the carboxyl side of the chromophore binding domain (starting at GRLEEVVAARTA . . . and ending at the end of the rcaE gene). The region of the cph1 gene from Synechocystis sp. PCC 6803 encoding the region containing the chromophore binding domain (starting with the single letter amino acid code MATTVQLSDQS . . . and ending at the sequence . . . VVFSNISAGED) will be amplified using PCR and covalently attached, using standard molecular biology techniques, to the portion of the rcaE gene from F. diplosiphon encoding the region of the protein beginning at the carboxyl side of the chromophore binding domain (starting at GRLEEVVAARTA . . . and ending at the end of the rcaE gene).

As is known by those of skill in the art, PixJ1 is a blue/green photoreversible photoreceptor that controls phototaxis (movement in response to light) in the cyanobacterium Synechocystis sp. PCC 6803, while Cph1 is a red/far red photoreversible photoreceptor of unknown function in the same organism. The advantages of these two photoreceptors are that they both: (a) have GAF domains that are very similar to the GAF domain found in RcaE; (b) contain these GAF domains in the same region of the protein as RcaE; (c) have had their covalently attached bilin identified; and (d) autocatalytically attach their bilin chromophore in E. coli (Gambatta and Lagarias 2001; Yoshihara et al. 2006) Because the domain structures of these three proteins are similar, replacing the chromophore attachment domain of RcaE with that of PixJ1 and Cph1 is useful for creating chimeric photoreceptors that attach a specific bilin in E. coli and operate as blue/green (for PixJ1) or red/far red (for Cph1) photoreceptors, yet are still capable of controlling RcaF and RcaC, and thus cpc2 promoter activity (see Gambatta and Lagarias 2001; Yoshihara et al. 2006).

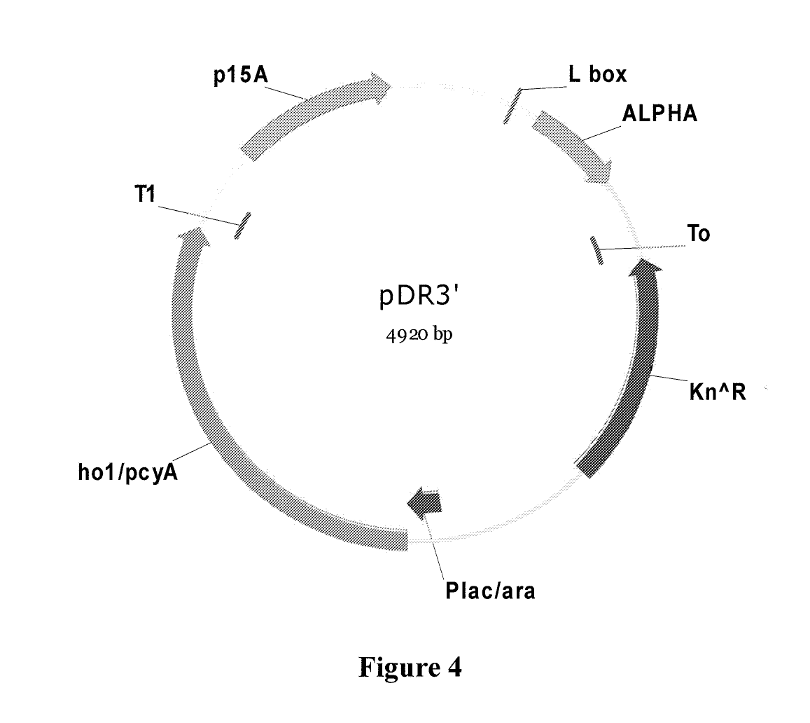

Regarding the covalent attachment of photoreceptor bilins to E. coli produced cyanobacterial photoreceptors, it has been generally discovered that this is an autocatalytic event that can occur in vitro using purified proteins and exogenously added chromophores, as well as in E. coli cells that are expressing the genes needed to make the two enzymes that convert heme to phycocyanobilin. It should be noted that E. coli does not naturally make the appropriate bilin chromophore, phycocyanobilin (PCB), which is used by PixJ1 and Cph1. However, it does make heme, which is the natural precursor of PCB. Addition of the two genes to E. coli that encode the enzymes heme oxygenase and phycocyanobilin oxidoreductase, which are necessary to covert heme to PCB, has been shown to be sufficient for its proper attachment to both PixJ1 and Cph1 (see Gambatta and Lagarias 2001; Yoshihara et al. 2006). As shown in FIG. 4, pDR3′, a plasmid containing the two genes that encode the enzymes heme oxygenase and phycocyanobilin oxidoreductase, has been created in a manner equivalent to that described in Gambatta and Lagarias 2001 and introduced into E. coli cells as part of the present teachings. It is highly certain that proper chromophore attachment will occur for PixJ1/RcaE and Cph1/RcaE chimeras as well, and thus they would serve as functional photoreceptors in E. coli, as PixJ1 and Cph1 have already been shown to do. Alternatively, PCB could be added exogenously to cells expressing these chimeric photoreceptors.

There are no problems anticipated with the production of the single-domain response regulator RcaF in E. coli. This protein has already been over expressed in E. coli so it is envisioned that it would also function correctly in this organism.

According to another exemplary embodiment, the synthesis of RcaC in E. coli is produced according to the present teachings. According to this embodiment, it is noted that one likely difference between the expression of RcaC in E. coli and in F. diplosiphon is that in the latter, RcaC is approximately about six times more abundant in red light grown cells than in green light grown cells (see Li and Kehoe 2005). While the biological significance of this finding is unclear (especially since it has been discovered that there is no significant change in RcaE levels between these two light conditions), it is expected that RcaC will still function correctly when expressed equally under all light conditions in E. coli, particularly as it has been found that it is the phosphorylation state of RcaC, rather than its abundance, that primarily affects its activity in F. diplosiphon (Li and Kehoe, unpublished results).

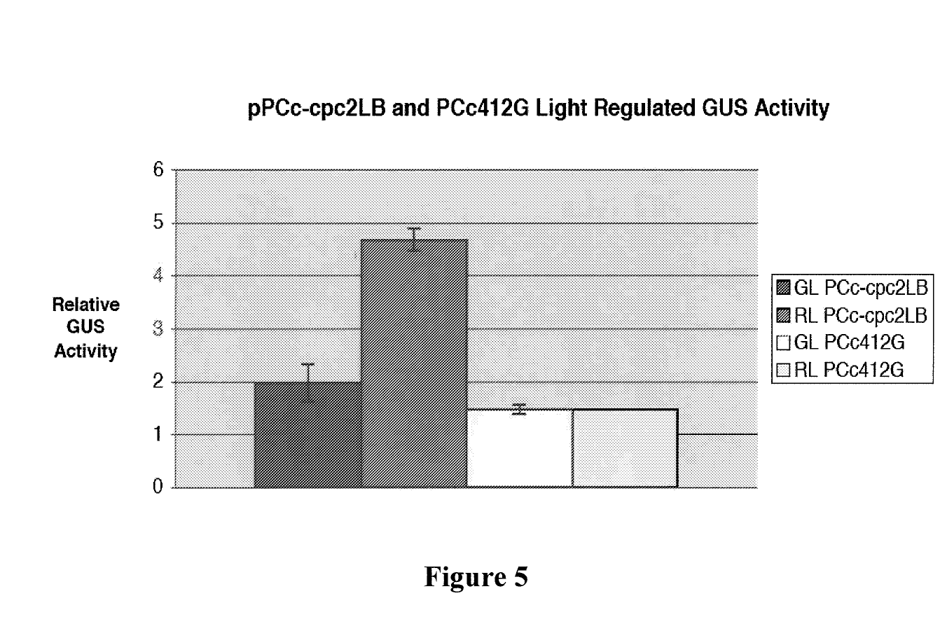

A critical and novel aspect of RcaC function is that it has been recently shown that it binds to a DNA element called the “L Box”, which consists of a direct repeat of the sequence 5′ TTGCACA 3′. The two repeats are separated by four base pairs. This element is present in the red light inducible promoters of the cpc2 and pcyA genes in both Synechococcus sp. PCC7335 and in F. diplosiphon, where it has been shown to be centered at −35 relative to the transcription start sites of these genes, and it has also been shown that RcaC binds specifically to these repeats in both of these promoters. In addition, the L Box has been shown to be both necessary and sufficient to confer red light regulation to gene expression. Replacement of the L Box with random sequence resulted in the loss of red light activation, but not overall expression, of the pcyA gene (Alvey and Kehoe 2006), and placement of the L Box from the cpc2 promoter into the normally non-light color regulated cpc1 promoter conferred red light regulated expression on cpc1 (Olcum, N., Bezy, R., and D. M. Kehoe, unpublished data). As shown in FIG. 5, data showing that the activity of the non-light responsive cpc1 promoter (PCc412G), when modified to include an L Box from the red light inducible cpc2 promoter (PCc-cpc2LB), becomes red light inducible.

The basal transcriptional machinery functions similarly in E. coli and cyanobacteria (Shibato et al. 2002). With respect to the degree to which the E. coli DNA-dependent RNA polymerase (RNAP) will recognize this particular cyanobacterial cpc2 promoter, it has been found that this must be determined empirically, particularly since some cyanobacterial promoters work very well in E. coli while others do not. Also, the activity of some promoters which initially failed to work in E. coli has been rescued by additionally providing the gene encoding a cyanobacterial RNAP alpha subunit.

In further exemplary embodiments of the present teachings, a system for regulating the transcriptional activity of target genes of prokaryotic organisms can be created in which RcaC functions as a repressor protein rather than an activator. As such, the present teachings are not intended to be limiting in nature.

Advantages and improvements of the processes and methods of the present invention are discussed in detail below and demonstrated in the following exemplary examples. These examples are illustrative only and are not intended to limit or preclude other embodiments of the invention.

INTRODUCTION: Phytochromes and phytochrome-like proteins in bacteria display tremendous structural diversity relative to their plant counterparts (Montgomery and Lagarias, 2002; Karniol et al., 2005). The responses these photoreceptors are known to control are also quite diverse. As in plants, prokaryotic phytochrome superfamily members control the synthesis of components of the photosynthetic apparatus, often via the transcriptional regulation of the corresponding genes (Kehoe and Grossman, 1996; Giraud et al., 2002; Jaubert et al., 2004; Katayama et al., 2004; Giraud et al., 2005), by regulating growth rates (Wilde et al., 1997; Fiedler et al., 2004; Oberpichler et al., 2006), and resetting of the circadian clock (Schmitz et al., 2000). In addition, they control phototaxis (Yoshihara et al., 2000; Bhaya et al., 2001; Wilde et al., 2002; Ng et al., 2003; Fiedler et al., 2005), chalcone synthase gene expression (Jiang et al., 1999), cAMP signaling (Ohmori et al., 2002), carotenoid synthesis (Davis et al., 1999a), and global gene expression changes (Hubschmann et al., 2005). This list is certain to grow substantially since the vast majority of these photoreceptors, which have been primarily identified through genome sequencing efforts, have not yet been assigned a function.

In addition, the downstream signaling pathways through which these photoreceptors operate are just beginning to be understood. The majority appear to function as histidine kinases and operate within two component regulatory systems. For some of these, the phosphoaccepting response regulator partner(s) has been identified by its genomic proximity to the photoreceptor and confirmed biochemically through the analysis of phosphotransfer reactions (Yeh et al., 1997; Bhoo et al., 2001; Giraud et al., 2005). However, the absence of output domains within these response regulators has precluded further characterization of these complex light regulated signaling pathways.

The most thoroughly characterized signal transduction pathway regulated by a bacterial phytochrome-class photoreceptor controls a response known as complementary chromatic adaptation (CCA) (Tandeau de Marsac, 1983; Grossman, 2003; Kehoe and Gutu, 2006). CCA occurs in many freshwater, marine, and soil-dwelling cyanobacteria. The physiology, molecular biology, and regulation of this process have been best described in the filamentous species Fremyella diplosiphon UTEX 481 (Calothrix/Tolypothrix PCC 7601). During CCA, the composition of cyanobacterial photosynthetic light harvesting structures, called phycobilisomes (PBS), is adjusted in response to changes in ambient light color, allowing efficient photon capture and photosynthetic activity over a range of light wavelengths. CCA is regulated at least in part by three known components that make up a complex two-component system called a phosphorelay. RcaE, a phytochrome-class photoreceptor, is also a member of the sensor histidine kinase family (Kehoe and Grossman, 1996). RcaF is composed of a single receiver domain and acts after RcaE (Kehoe and Grossman, 1997). RcaC acts after RcaF and contains two receiver domains, a histidine phosphotransfer domain, and an OmpR/PhoB DNA binding domain (Chiang et al., 1992; Kehoe and Grossman, 1997).

In F. diplosiphon, PBS consist of an inner core that is associated with the thylakoid membrane and six “rods” radiating outward from the core that are responsible for capturing and transferring light energy through the core to photosynthetic reaction centers (MacColl, 1998). They contain proteins with (pigmented) and without (non-pigmented) covalently attached chromophores. The major pigmented proteins in PBS consist of blue colored allophycocyanin [AP; absorption maximum (Aₘₐₓ)=650 nm] and phycocyanin (PC; Aₘₐₓ=620 nm), and red colored phycoerythrin (PE; Aₘₐₓ=565 nm). AP is present in the core, while the rods contain PC and sometimes PE. In addition, linker proteins, which are predominantly non-pigmented, are present in both cores and rods and have both structural and energy transfer roles (Glazer, 1989).

CCA in F. diplosiphon involves adjusting PBS composition by modulating the synthesis of both PC and PE in response to red light (RL) and green light (GL). PC accumulates to higher levels in RL and PE to higher levels in GL. Thus far, these changes have been found to be controlled transcriptionally and CCA regulation studies have largely focused on understanding CCA control of genes encoding PBS structural components (Oelmüller et al., 1988). Most cyanobacterial species capable of CCA possess at least two separate operons encoding PC (Bryant, 1981). In F. diplosiphon the cpc1 operon encodes PC1, which is present in the core proximal regions of the rods. PC1 abundance is not CCA regulated. The cpc2 operon encodes a second form of PC (PC2) and its associated linkers, which are incorporated into the core distal portions of the rods. Its expression is very high during growth in RL and virtually absent during growth in GL (Conley et al., 1985; Conley et al., 1986; Lomax et al., 1987). The genes encoding the major GL-specific PBS proteins in F. diplosiphon are cpeCDE, which encode the PE linker polypeptides, and cpeBA, which encode the PE proteins (Mazel et al., 1986; Federspiel and Grossman, 1990; Federspiel and Scott, 1992; Glauser et al., 1992). These two gene sets are unlinked in the genome and their expression is apparently coordinated through the initial GL activation of the operon containing the cpeCDE genes, cpeCDESTR. The key component within this operon for the subsequent activation of cpeBA is CpeR, which has been found to be both necessary and sufficient to induce cpeBA expression (Cobley et al., 2002; Seib and Kehoe, 2002).

The cis-acting promoter elements through which the Rca system operates have not yet been defined. However, one promoter-gusA reporter gene fusion study demonstrated that the CCA-responsive region of the cpc2 promoter was between −76 and +25 (relative to transcription start), a region that contains a 13 base pair direct repeat (Casey and Grossman, 1994). Further analysis of this repeat was not conducted, since removal of additional cpc2 promoter sequence resulted in undetectable GUS activity. No functional studies of the promoters of GL activated genes have been conducted.

CCA involves changes in the synthesis of both light harvesting apo-proteins and the chromophores that are covalently attached to them (Alvey et al., 2003). Open chain tetrapyrroles, or bilins, are the chromophores used by cyanobacteria for photosynthetic light harvesting. AP and PC use phycocyanobilin (PCB), while PE uses phycoerythrobilin (PEB) (Ó Heocha, 1963; Ó Carra et al., 1964; Chapman et al., 1967; Cole et al., 1967; Crespi et al., 1967). Bilin synthesis pathways have been characterized biochemically (Beale, 1993) and more recently genes encoding bilin biosynthetic enzymes have been identified and isolated from plants and cyanobacteria. These include the genes encoding heme oxygenase, HY1 in plants and ho1 in cyanobacteria, and phytochromobilin synthase, HY2, from plants (Cornejo et al., 1998; Davis et al., 1999b; Muramoto et al., 1999; Kohchi et al., 2001). In cyanobacteria, pcyA, which encodes the enzyme that catalyzes the four electron reduction of biliverdin IXα (BV) to PCB, and pebA and pebB, which encode proteins that carry out the four electron reduction of BV to PEB, have also been cloned and characterized (Cornejo and Beale, 1997; Frankenberg et al., 2001; Alvey et al., 2003; Frankenberg and Lagarias, 2003; Tu et al., 2004; Dammeyer and Frankenberg-Dinkel, 2006; Hagiwara et al., 2006a, b; Tu et al., 2006).

Both physiological and molecular data have shown that during light harvesting antennae biogenesis, apo-protein and chromophore synthesis is tightly coordinated (Troxler, 1972; Anderson and Toole, 1998; Toole et al., 1998; Alvey et al., 2003). However, the mechanisms through which this coordination is achieved remains largely unexplored. Bogorad (1975) suggested that the three most likely ways of controlling such co-production are: (a) chromophore production dictates the synthesis of corresponding apo-protein, (b) apo-protein production dictates the synthesis of the appropriate chromophore, or (c) apo-protein and chromophore production are regulated in parallel through a common mechanism. With the identification of the pcyA, pebA, and pebB genes, this issue has begun to be addressed. In F. diplosiphon, expression of the pebAB operon was greater in GL than RL, similar to cpeBA and cpeCDE. Its expression was also dependent on CpeR, suggesting that in F. diplosiphon, coordinated expression of the cpeBA and pebAB operons occurs in parallel through the same mechanism (Alvey et al., 2003). This coordination was proposed to occur through a common DNA sequence element that is present in the promoters of pebAB and cpeBA. Although this element, which was named the N Box, previously has been shown to be a protein binding site (Schmidt-Goff and Federspiel, 1993; Sobczyk et al., 1993), no functional studies have been conducted on it, and thus its role in CCA regulation remains unknown.

Additional features of the CCA control system have been investigated by extending the analysis of bilin biosynthetic gene regulation to the pcyA gene in F. diplosiphon. It was determined that pcyA was more highly expressed in RL than GL and is CCA regulated through the Rca signal transduction pathway. A novel direct repeat DNA sequence within the pcyA promoter was identified that was also present at a nearly identical position within the F. diplosiphon cpc2 promoter, thereby suggesting that the CCA regulation of cpc2 and pcyA likely occur through a common mechanism. Mutation of this element within the pcyA promoter led to a loss of RL induction, demonstrating its central role in the CCA regulation of RL induced genes. This is the initial cis-acting DNA element to be directly implicated in the regulation of CCA and of any prokaryotic two-component pathway controlled by a phytochrome family photoreceptor. Interestingly, it was also found that this direct repeat within the promoter of the GL induced cpeCDESTR operon, thereby strongly suggesting that this CCA-responsive element plays a central role in coordinating the expression of RL and GL induced genes during CCA.

According to this exemplary illustration, the mechanisms underlying the light regulation of pcyA, the gene encoding phycocyanobilin:ferredoxin oxidoreductase, in the filamentous cyanobacterium Fremyella diplosiphon was investigated. pcyA expression is controlled by the well characterized Rca system, a complex phosphorelay whose activity is modulated by the phytochrome-class photoreceptor RcaE. This system controls both the activation and repression of genes by red light during a process called complementary chromatic adaptation. The expression of pcyA appears to be under the control of two promoters, one that is red light activated and one that is light-color insensitive. The red light inducible promoter contains a regulatory element named the L Box that is also present within the promoter of the red light induced cpc2 operon, which encodes phycocyanin apo-proteins and associated linker proteins. The L Box is required for the red light induction of pcyA expression and thus has a central role in coordinating phycocyanin apo-protein and chromophore synthesis. A differently positioned L Box was also identified within the red light repressed cpeCDESTR promoter. Thus, this element may be broadly important for coordinating the activation and repression of RcaE controlled genes.

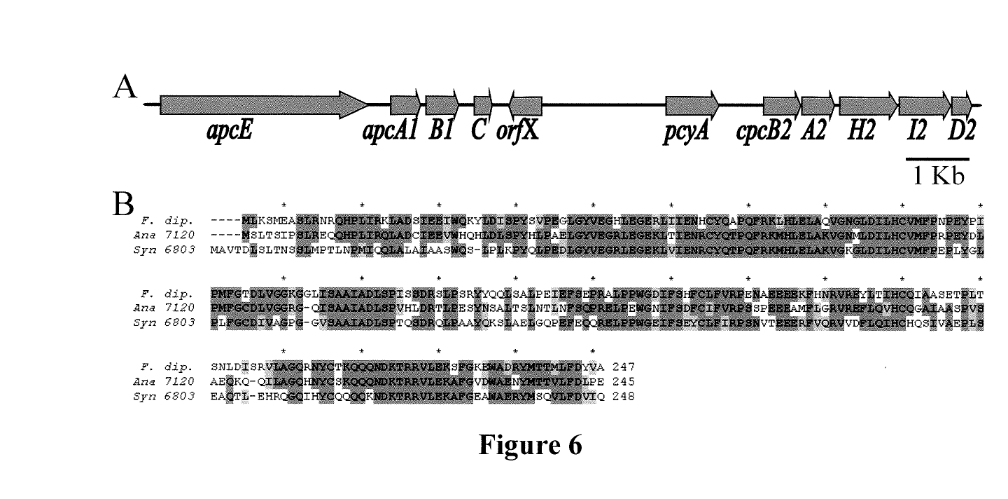

RESULTS: A DNA fragment containing the presumed pcyA gene from F. diplosiphon was cloned using primers based on conserved pcyA sequences from several other cyanobacterial species and PCR amplification. Sequence analysis revealed an open reading frame (ORF) encoding a putative PcyA protein 247 amino acids in length. This gene was situated between the apc operon, encoding the AP core components of the PBS, and the cpc2 operon (FIG. 6A) (Conley et al., 1986). More particularly, FIG. 6A shows relative positions and orientations of the genes/ORF surrounding pcyA, wherein the arrowhead indicates the direction of transcription and 1 Kb bar represents 1 kbp of DNA. Based on restriction mapping data, the cpc2 and apc operons had been reported previously to be in proximity in F. diplosiphon (Conley et al., 1986). The translated protein sequence from this ORF was 65% identical and 80% similar over 237 amino acids to the PcyA protein from Anabaena sp. PCC 7120 and 55% identical and 71% similar over 245 amino acids to PcyA from Synechocystis sp. PCC 6803 (Frankenberg et al., 2001) (FIG. 6B). More particularly, FIG. 6B shows the complete amino acid sequence of F. diplosiphon (F. dip.) pcyA aligned with pcyA genes from Anabaena sp. PCC 7120 (Ana 7120) and Synechocystis sp. PCC 6803 (Syn 6803), wherein the darker gray boxes highlight positions of amino acid identity, and the lighter gray boxes highlight positions of similarity. The number provided in FIG. 6B indicates the amino acid position, relative to the first methionine, shown for each sequence. Other than the putative pcyA gene, only one ORF of more than 100 codons (ORF X) was present in the 4.1 kbp between the apc and cpc2 operons (FIG. 6). It is encoded on the opposite DNA strand from the putative pcyA gene and the 134 amino acids potentially encoded by this ORF have no significant similarity to any sequences in GenBank (data not shown). The sequence of these ORFs and the flanking DNA were confirmed by isolating and sequencing a clone containing this region from a F. diplosiphon genomic DNA phage library.

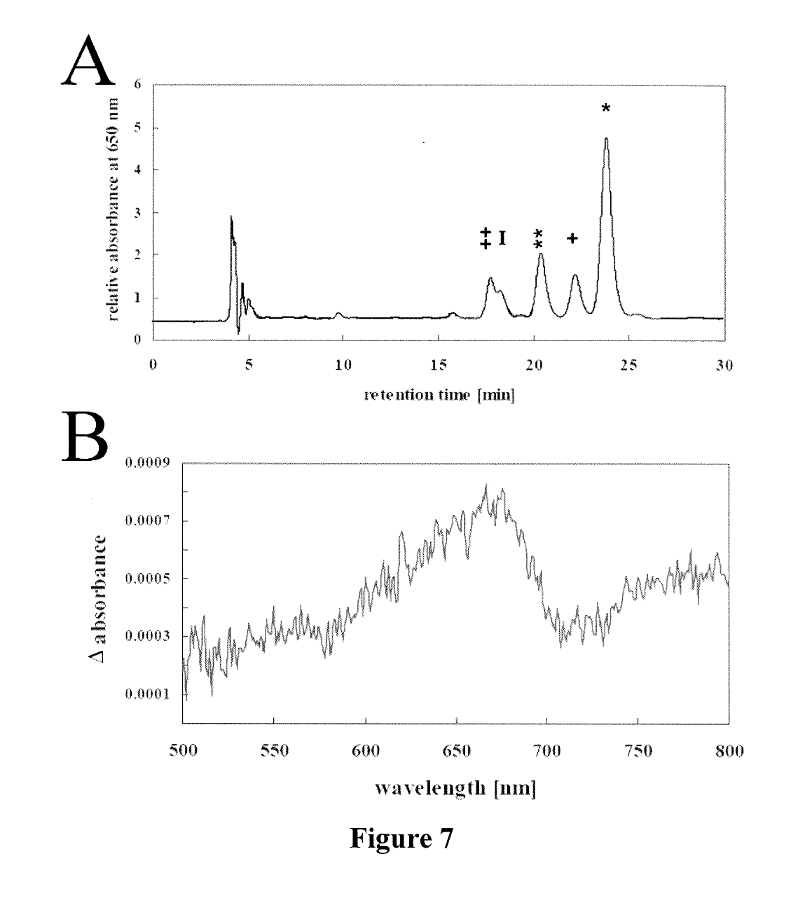

It was established that this putative pcyA gene encoded a functional phycocyanobilin:ferredoxin oxidoreductase by expressing it in Escherichia coli, partially purifying it, and in vitro testing of extracts containing this protein for PCB production from exogenously supplied BV by reverse phase HPLC separation and spectrophotometric detection. When extracts from cells expressing the putative pcyA gene were used, absorption products eluted at the times consistent with the isomers 3E-PCB and 3Z-PCB (FIG. 7A). More particularly, FIG. 7A demonstrates that F. diplosiphon PcyA converts BV to PCB as detected by HPLC. A soluble protein extract from IPTG induced E. coli cells carrying pMal_pcyA was assayed for bilin synthase activity. Bilins were extracted from the protein sample and analyzed by reversed phase HPLC. The eluate was monitored at 650 nm. Known bilin standards eluted with the following elution times: 3E-PCB (17.8 min), 18¹,18²-dihydrobiliverdin (20.4 min), 3Z-PCB (22.2 min) and BV IXα (23.9 min). Symbols used are BV IXα (*); 18¹,18²-DHBV (**); 3E-PCB (++); 3Z-PCB (+). I is a known impurity in the commercially available BV IXα.

Such products have not been observed in E. coli cells not expressing pcyA (Frankenberg et al., 2001). That PCB was produced in these extracts was further confirmed by adding apo-Cph1, a Synechocystis sp. PCC 6803 phytochrome photoreceptor, and measuring the red-far red-light induced difference spectrum for the sample. The spectrum obtained was essentially identical to those obtained in previous studies that used purified PCB for in vitro attachment to apo-Cph1 (FIG. 7B) (Frankenberg et al., 2001; Frankenberg and Lagarias, 2003). More particularly, FIG. 7B shows the phytochrome difference spectrum of Cph1 incubated with MBP-PcyA bilin reductase assay reaction products. According to this figure, a soluble protein extract of IPTG-induced E. coli JM109 carrying pMal_pcyA was assayed for bilin synthase activity as described in the present methods. Taken together, these data demonstrate that this F. diplosiphon gene is pcyA.

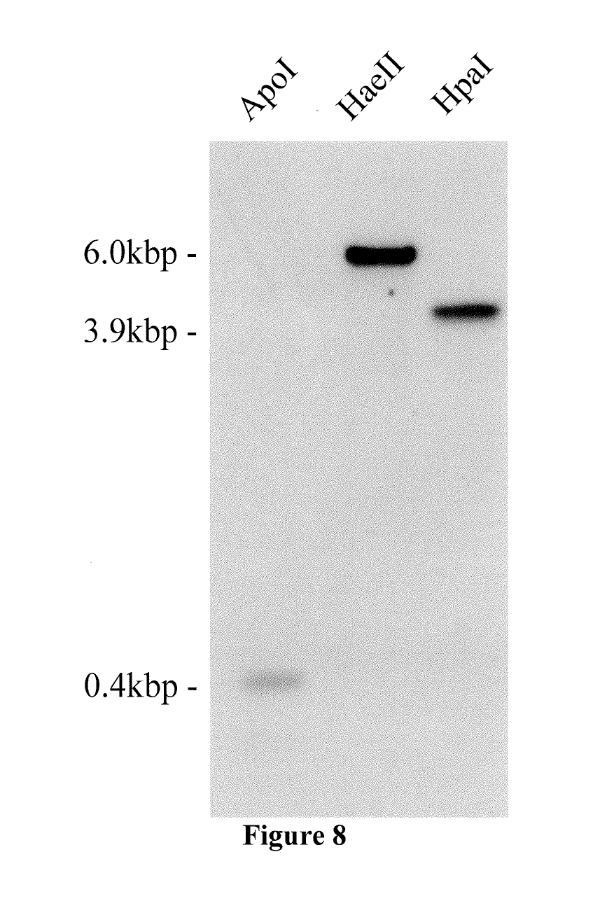

The presence of multiple operons encoding PC in F. diplosiphon suggested that this organism might also contain multiple pcyA genes, for example, one that was RL activated and another whose expression was not light regulated. Southern blot analysis was used to determine if any other sequences closely related to the isolated pcyA gene were present in the F. diplosiphon genome. The results indicated that pcyA was only present in a single copy in this organism (see FIG. 8-Autoradiogram of a membrane containing F. diplosiphon genomic DNA cut with the restriction endonucleases Apo I, Hae II, and Hpa I and probed with a ³²P-labeled F. diplosiphon pcyA DNA fragment, wherein DNA lengths are indicated on the left).

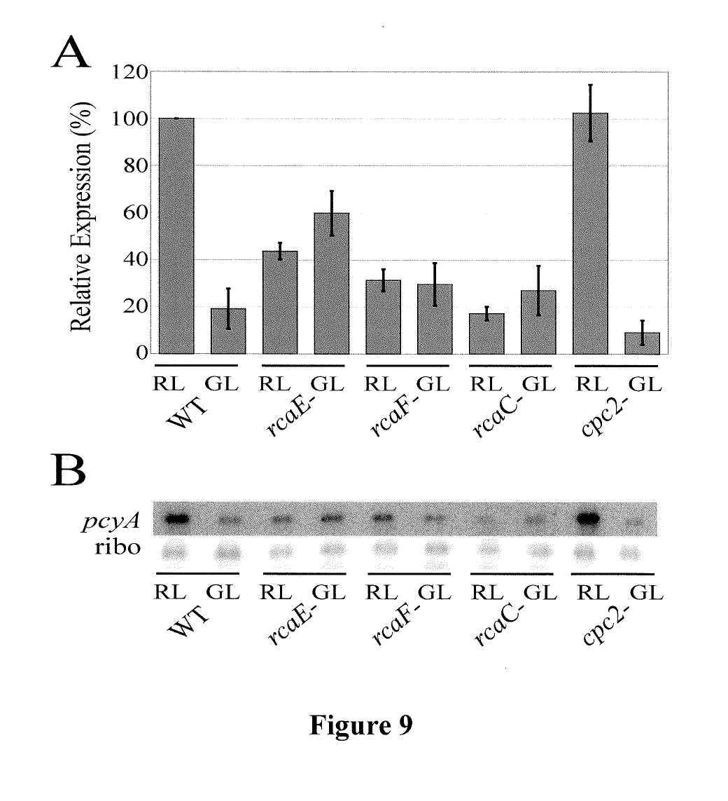

To characterize pcyA transcript accumulation patterns and test whether pcyA was CCA regulated, RNA gel blot analyses were conducted using wild type and CCA regulatory mutant cells grown in RL and GL. In wild type cells, pcyA mRNA accumulated to a level nearly five times higher during growth in RL compared to growth in GL (FIG. 9). It was noteworthy that, unlike for the cpc2 operon, pcyA mRNA still accumulated to a significant level during growth in GL. The possibility that the RL induced increase in pcyA mRNA was under CCA control was tested by conducting the same analysis using mutants with lesions in the CCA regulatory genes rcaE, rcaF, and rcaC. In each of these mutants, pcyA mRNA levels were not significantly regulated by light color. These results demonstrate that each of these genes is required for such regulation and that pcyA is under CCA control (FIG. 9).

Specifically, FIG. 9A shows a summary of data collected from three independent RNA blot analyses using a fragment of the F. diplosiphon pcyA gene as a probe. RNA levels are expressed as a percentage of the level of pcyA transcript measured in wild type cells grown in RL, which was set to 100. Before the calculation of the means, all values were normalized for loading errors using relative ribosomal hybridization values as previously described (Seib and Kehoe 2002). Error bars denote standard errors of the means. Results obtained for wild type, three different Rca insertion mutants, and a cpc2 insertion mutant are shown. FIG. 9B shows an autoradiogram of one of the RNA blots used to obtain the results presented in FIG. 9A after hybridization with a F. diplosiphon pcyA probe, wherein “ribo” refers to the autoradiogram results obtained from the same blot after stripping and reprobing with a ribosomal probe.

The possibility still existed that the co-induction of cpc2 and pcyA occurred through a feedback mechanism in which the expression of pcyA required the expression of cpc2. This was addressed by using RNA blot analysis to examine pcyA mRNA levels in a cpc2 null mutant (R. Alvey and D. Kehoe, unpublished results). In this mutant, pcyA expression was nearly the same as that measured for wild type cells during growth in both RL and GL (FIG. 9). This demonstrates that the CCA regulation of pcyA expression is direct and does not involve a feedback control mechanism that requires cpc2 production.

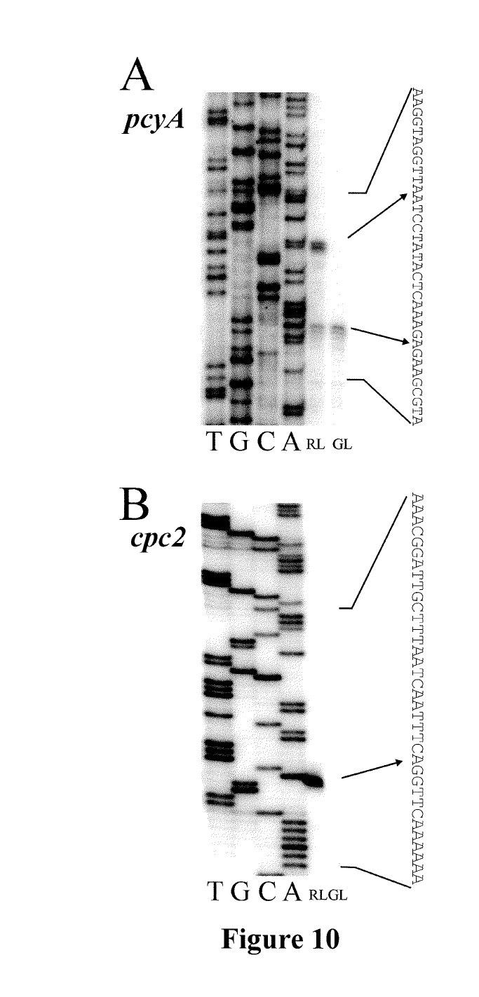

The transcription initiation site(s) for pcyA in F. diplosiphon were mapped using a ribonuclease protection assay (RPA) and RNA isolated from wild type cells grown in RL and GL. A protected product of identical length and abundance was generated from RNA samples isolated from both light conditions (FIG. 10A). As shown in FIG. 10A, the transcription start sites were determined during growth in RL and GL for pcyA using a ribonuclease protection assay and as shown in FIG. 10B, for cpc2 by primer extension analysis. For each, autoradiograms show the DNA sequencing reaction products in the four left lanes and the start site analysis products in the right two lanes. Arrows indicate the initial (5′-terminal) bases of the analyzed transcripts. Results shown in (A) and (B) are typical of those obtained from three independent RNA isolations and analyses.

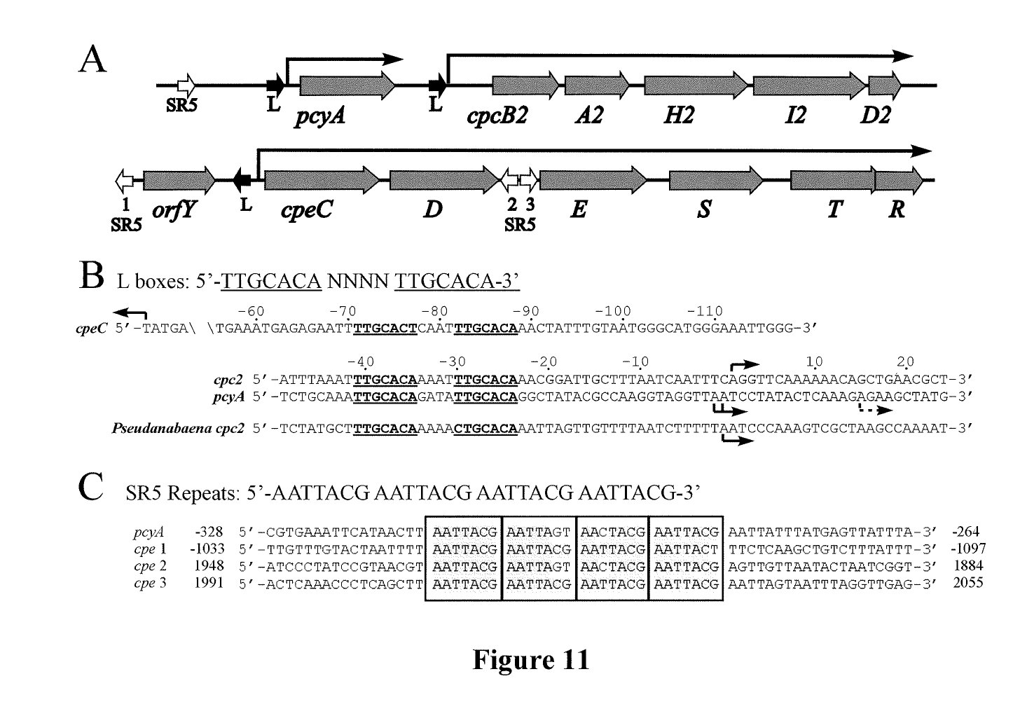

As can be seen from FIG. 11, the F. diplosiphon pcyA promoter region shares common elements with other CCA regulated genes. More particularly, FIG. 11A shows the relative positions of potential regulatory elements called STRR5 repeats (“SR5”) (denoted by small white arrows) and L Boxes (“L”) (denoted by small black arrows) within the regions near pcyA and cpeCDESTR. The gray boxes represent the genes/ORFs, while the direction of transcription is indicated by the arrowhead. Each thin black arrow demarks a region that is continuously transcribed. The three STRR5 repeats in and around the cpeCDESTR operon have been labeled “1-SR5, 2-SR5, and 3-SR5” for reference purposes. The orientations of all potential regulatory elements, relative to those in the pcyA promoter region, are indicated by the direction of the white arrow.

FIG. 11B shows sequence alignments of L Boxes (consensus sequence provided at top) within the light regulated promoters of three F. diplosiphon genes and the cpc2 gene of Pseudanabaena sp. PCC 7409. Upper and lower numberings are relative to transcription start sites of the F. diplosiphon cpeCDESTR and cpc2 operons, respectively. Note that the cpeCDESTR L Box is in the opposite orientation relative to the others. Bent arrows denote transcription start sites. The thin arrow identifies the GL-specific start site of cpeCDESTR, the bold arrows show RL-specific start sites, and the dashed line designates the non-CCA regulated start site of pcyA, which is eight bases 5′ of the predicted ATG start codon. FIG. 11C shows alignment of the F. diplosiphon STRR5 repeats sequences (consensus sequence provided at top). Each repeat is framed, and the presence of the consensus nucleotide at each position within each repeat is indicated by a gray box. The numbering provided is relative to the transcription start site of either pcyA or cpeCDESTR.

The length of this product places the predicted transcription start site for this transcript eight by upstream of the apparent pcyA start codon (FIG. 11B). A second, longer protected product was also identified exclusively from the RL RNA sample. The corresponding start site of this transcript was 24 by upstream of the start codon. These results suggest that there are two transcription start sites for pcyA in F. diplosiphon, and that the initiation of transcription at one is regulated by CCA while at the other it is not light regulated.

Several different transcription start sites have been reported for the cpc2 operon in F. diplosiphon (Conley et al., 1988; Sobczyk et al., 1994; Liotenberg et al., 1996). In order to facilitate the analysis and comparison of the pcyA and cpc2 promoters, primer extension assays were conducted in order to determine the transcription start site for cpc2. RNA samples from wild type cells grown in RL and GL were used for this study. A single extension product was obtained from the RNA isolated from RL grown cells, while no product was obtained using RNA from GL grown cells (FIG. 10B). The start site predicted from this analysis was the same as the “P1” site identified previously (Liotenberg et al., 1996). This was located 351 by upstream of the cpcB2 start codon (Conley et al., 1985; Conley et al., 1988).

The region of DNA upstream of the two pcyA transcription initiation sites was compared to promoter sequences of other CCA regulated genes from F. diplosiphon in an effort to identify shared DNA motifs that might have a common role in their regulation during CCA. Two candidate elements were identified (FIG. 11A). The first, which is designated the “L Box,” consists of a direct repeat of the sequence 5′-TTGCACA-3′, separated by four nucleotides (FIG. 11B). It is also contained within the 13 nucleotide overlapping direct repeat of 5′-AAATTTGCACAAA-3′ that has been previously noted as a potential regulatory element within the cpc2 promoter (Casey and Grossman, 1994). This element is positioned nearly identically in the cpc2 and pcyA promoters, centered at 32 by and 30-31 by upstream of their RL-dependent transcription start sites, respectively, as shown in FIG. 10B. In addition, an L Box was discovered within the promoter of the GL activated cpeCDESTR operon. This L Box differed by a single nucleotide and was oriented, relative to cpeCDESTR, in the opposite direction from those within the pcyA and cpc2 promoters (FIG. 11B). Its location was also different, being centered 80 by upstream of the cpeCDESTR transcription start site (Federspiel and Grossman, 1990) (R. Bezy and D. Kehoe, unpublished results).

Thus far, there is only one other cpc2 operon with a mapped transcription start site whose expression has been shown to be RL inducible, that of Pseudanabaena sp. PCC 7409 (Dubbs and Bryant, 1993). The promoter region of this operon was examined in order to determine whether an L Box was also present in this strain. It was found to possess an L Box (FIG. 11B), present in the same orientation as the pcyA and cpc2 L Boxes in F. diplosiphon, and its sequence was also identical except for a single nucleotide change. In addition, alignment of the transcription start sites of these RL-responsive promoters revealed that the cpc2 L Box in Pseudanabaena was centered 31 by upstream of the transcription start site, nearly the identical position as the L Boxes within the cpc2 and pcyA promoters of F. diplosiphon.

The second sequence identified upstream of pcyA consists of four tandem copies of 5′ AATTACG 3′ (FIG. 11C). This seven by sequence already has been noted to occur at a high frequency in other cyanobacteria. It is one of a class of short tandemly repeated repetitive (STRR) sequences of unknown function and was previously designated STRR5 (Mazel et al.; Meeks et al., 2001). An STRR5 repeat is centered at 297 by upstream of the transcription start site of pcyA (FIGS. 11A and 11C). This sequence was not identified immediately upstream of cpc2, but three copies were found near the cpeCDESTR operon (FIGS. 11A and 11C). The first of these was centered 253 by upstream of the hypothetical translation start site of ORF Y, located upstream of cpeC, which places it centered 1,063 by upstream of the transcription initiation site of cpeCDESTR (Federspiel and Grossman, 1990; R. Bezy and D. Kehoe, unpublished results). The other two STRR5 repeats are located within the cpeCDESTR operon in the intergenic region between cpeD and cpeE, positioned in opposite orientations and separated by 76 by (FIGS. 11A and 11C).

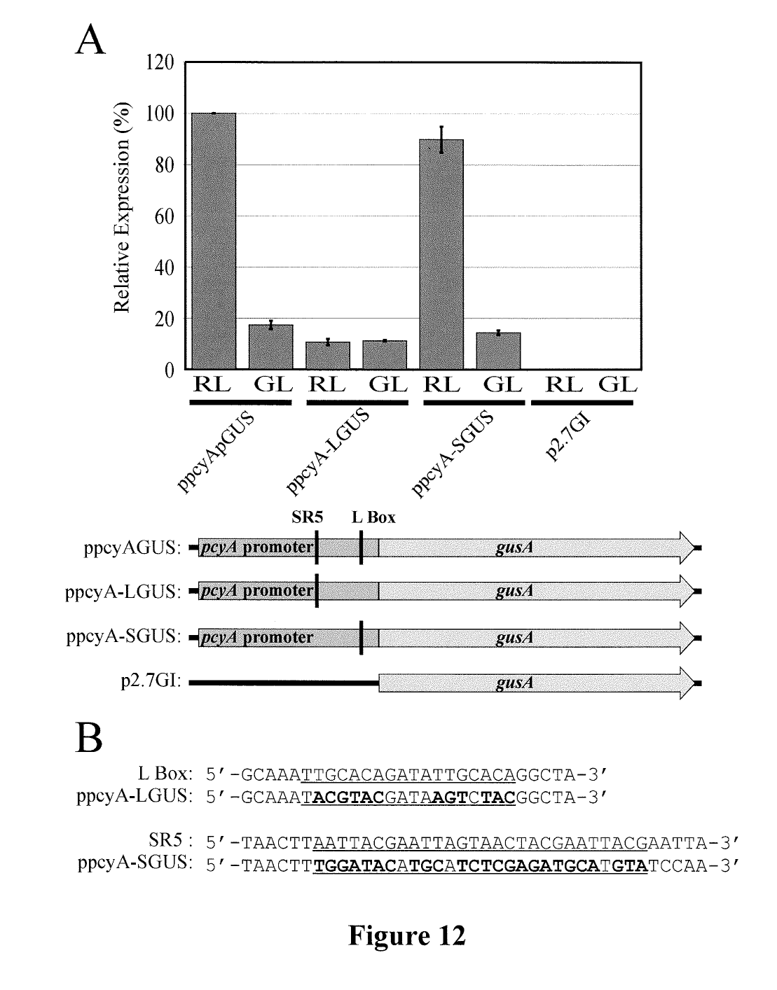

The presence of the L Boxes and STRR5 repeats near both RL and GL activated genes suggested the possibility that these elements were involved in CCA transcriptional regulation. This was tested using the region upstream of pcyA by creating a series of reporter gene constructs. The region of pcyA from −1000 to +25, relative to the apparent downstream transcription start site, was joined to the promoterless gusA gene (encoding β-glucuronidase, or GUS) within the autonomously replicating plasmid p2.7GI (Casey and Grossman, 1994). The new plasmid was named ppcyApGUS. It was transformed into wild type F. diplosiphon cells, which were then grown in both RL and GL. Extracts from these cells were then tested for GUS activity. These transformants showed an approximately five-fold higher level of GUS activity in extracts from RL grown cells than from GL grown cell extracts (FIG. 12A). More particularly, FIG. 12A shows relative rates of GUS activity per mg protein in extracts derived from F. diplosiphon cells transformed with the indicated plasmids and grown in either RL or GL prior to harvest. The mean value derived from cells transformed with ppcyApGUS and grown in RL was set at 100%. This value was 46.3 nmol of product per mg of protein per min. Three independently transformed lines were tested for each plasmid. Error bars show the standard error of the mean.

This degree of induction was essentially identical to that measured previously for pcyA mRNA (FIG. 9A), demonstrating that CCA regulation of pcyA expression is at the transcriptional level and that this 1,024 by DNA fragment from upstream of pcyA was capable of conferring CCA responsiveness to the gusA reporter gene. In addition, the pcyA promoter was clearly active during growth in GL, since GUS activity was significantly higher in these transformants than the GUS activity in wild type cells transformed with p2.7GI (FIG. 12A).

The possible role(s) of the L Box and STRR5 repeats in mediating the CCA response of pcyA were examined by constructing two substitution mutants (FIG. 12B). More particularly, FIG. 12B shows sequence alignments of the pcyA wild type L Box and STRR5 repeat sequences and the sequences used to replace them (both underlined) in ppcyA-LGUS and ppcyA-SGUS. Actual changes within these repeats are shown in bold. In the first, the two seven by repeats 5′ TTGCACA 3′ within the L Box of ppcyApGUS were replaced with an identical number of base pairs of randomly generated sequence. This plasmid was named ppcyA-LGUS. In the second, each of the four repeats within the STRR5 sequence was replaced with an identical number of base pairs of randomly generated sequence, creating the plasmid ppcyA-SGUS. Each construct was transformed into wild type cells, grown in RL and GL, and tested for GUS activity (FIG. 12A). Replacing the L Box resulted in the complete loss of CCA regulation of GUS activity due to the loss of activity in RL. GUS activity under both light conditions was slightly below the level measured for cells transformed with ppcyApGUS and grown in GL but still significantly above the level for p2.7GI transformants grown in either RL or GL, which was essentially undetectable (FIG. 12A). These results clearly demonstrate that the L Box within the pcyA promoter is required for CCA responsiveness, since there was basal promoter activity but no RL induction for the L Box deletion plasmid. A similar analysis of wild type cells transformed with ppcyA-SGUS demonstrated that the replacement of the STRR5 repeats sequence had no effect on the CCA regulation of GUS activity, although these transformants did have a slightly lower level of GUS activity than the positive control during growth in both RL and GL (FIG. 12A). These results demonstrate that the L Box is necessary for the pcyA CCA response, while the STRR5 repeats are not.

DISCUSSION: The analysis of pcyA expression and its control in F. diplosiphon provides additional insight into signaling mechanisms through which members of the bacterial phytochrome superfamily operate. In addition, it contributes significant new information on the mechanisms of CCA regulation and how apo-protein and chromophore synthesis is coordinated in cyanobacteria.

Both cpc2 and pcyA are most highly expressed during growth in RL. However, unlike cpc2, pcyA mRNA accumulation also occurs during growth in GL, a finding that might have been predicted based on the fact that both AP and PC1 are synthesized during GL growth and use PCB as their chromophore (Conley et al., 1986). Although F. diplosiphon could have utilized two separate pcyA genes in a manner similar to cpc1 and cpc2, with one transcribed constitutively and one expressed only in RL, this arrangement did not arise. The expression of the single pcyA gene can be most simply explained by the existence of by two tandem promoters, an upstream one that is exclusively RL activated and a downstream one that is equally active under RL and GL (FIG. 10), although transcript processing cannot be ruled out from the data provided here.

It might be expected that the CCA regulated pcyA gene would be located within a gene cluster encoding PBS components in the F. diplosiphon genome, including the cpc2 operon (FIG. 6), since clustering of genes encoding proteins with linked functions is common in bacteria. However, it is somewhat surprising that this arrangement is not found in any of the cyanobacterial genomes that have been completely sequenced thus far. This genomic analysis revealed that the pcyA gene is not closely linked to genes encoding other photosynthetic components in any of 13 species with sequenced genomes, including Synechocystis sp. PCC 6803, Anabaena sp. PCC 7120, Thermosynechococcus elongatus BP-1, Gloeobacter violaceus PCC 7421, Prochlorococcus marinus (SS120, MED4, and MIT9313), Synechococcus sp. WH 8102, Synechococcus elongatus PCC 7942, Nostoc punctiforme ATCC29133, Anabaena variabilis ATCC29413, Crocosphaera watsonii WH8501, and Trichodesmium erythraeum IMS101 [Cyanobase (http://www.kazusa.or.jp/cyanobase/); JGI Microbial Genomics (http://genome.jgi-psforg/mic_home.html)]. The reason for the co-localization of pcyA and cpc2 in F. diplosiphon is not clear, but it is possible that such an arrangement facilitates the lateral transfer of components required for bestowing CCA regulation to PC expression. The existence of two different types of chromatic adaptation may support this possibility. Some cyanobacteria regulate only their PE accumulation in response to light color (“group II” chromatic adapters) while others, such as F. diplosiphon, regulate both PE and PC during chromatic adaptation (“group III” chromatic adapters) (Tandeau de Marsac, 1977). Group III strains may have arisen from group II by lateral gene transfer of RL activated genes such as pcyA and cpc2. If so, the clustering of these genes would have made it much easier to acquire group III capabilities. However, if this is true, either this process was not universally used during the development of all group III strains or there is not a very strict arrangement of pcyA and cpc2 positioning in such strains, since in the group III species Pseudanabaena sp. PCC 7409, the gene immediately upstream of cpc2 is not pcyA (Dubbs and Bryant, 1993). The relative locations of pcyA and cpc2 in this species have not yet been determined.

The RL induction of pcyA and cpc2 expression is transcriptionally controlled (FIG. 12) (Casey and Grossman, 1994), apparently through equivalent promoter elements (FIGS. 11 and 12), by the Rca system (FIG. 9). Additionally, there is virtually no change in pcyA activity in the absence of cpc2 expression (FIG. 9), demonstrating that at least the lack of cpc2 RNA and protein does not affect pcyA transcript abundance through a feedback regulatory mechanism. These results closely parallel those obtained for the coordinate regulation of cpeBA and pebAB in F. diplosiphon, which also occurs at the transcriptional level rather than through a feedback control system that senses the absence of RNA from either of these operons (Alvey et al., 2003). Thus, the general trend that is emerging for achieving coordination of PBS protein and bilin biosynthetic enzyme gene expression during CCA in cyanobacteria is via “top down” transcriptional regulatory systems rather than through feedback mechanisms.

However, although the transcription of all of the above genes is CCA regulated, the specific signaling mechanisms controlling their CCA responses appear to be different. This is supported by the following facts: first, the direct repeat sequence (N Box) present in the promoters of cpeBA (Sobczyk et al., 1993) and pebAB (Alvey et al., 2003) is also present in these promoters in a non-group III strain (Anderson and Grossman, 1990); second, the N Box sequence is quite different from that of the L Box and thus is unlikely to be bound by the same transcription factor; third, the expression of the cpeBA and pebAB operons is CpeR dependent, while for at least cpc2, it is not (Cobley et al., 2002; Seib and Kehoe, 2002); fourth, the N Box has been reported to be bound by a protein in either a GL-dependent or light independent manner, while a short region containing the L Box binds a protein in a RL-dependent fashion (Schmidt-Goff and Federspiel, 1993; Sobczyk et al., 1993; Casey and Grossman, 1994); fifth, the Rca system appears to completely control the CCA responses of pcyA and cpc2, yet only partially controls the CCA response for cpeBA and pebAB (Seib and Kehoe, 2002; Alvey et al., 2003).

CCA regulation of pcyA clearly does not depend on the presence of the STRR5 repeats (FIG. 12A). This sequence has been previously identified as occurring frequently within the genomes of other filamentous cyanobacterial species, including N. commune UTEX 584, N. punctiforme strain ATCC 29133, and Anabaena PCC 7120 (Angeloni and Potts, 1994; Meeks et al., 2001). Although STRR5 is overly represented in the genomes of filamentous, heterocyst-forming cyanobacteria, its abundance varies significantly between species, with 45 identical sites in N. punctiforme and only eight in Anabaena (Meeks et al., 2001). The function(s) of STRR5 and related repeat elements remains unknown.

To determine whether the L Box sequence was also overrepresented in the genomes of filamentous cyanobacteria, over 230,000 by of sequenced regions of the F. diplosiphon genome were analyzed, including the regions of the genome that have been shown to encode seven additional RL activated genes (Stowe-Evans et al., 2004), and the entire N. punctiforme (a group II chromatic adapting strain) genome for additional L Boxes. No L Boxes having a 90% or greater identity in these sequences (data not shown) could be identified. Thus, the only other L Box identified thus far is within the cpc2 promoter of Pseudanabaena sp. PCC 7409, making it likely that this element is highly specific for CCA regulation in multiple cyanobacterial species. It is not clear why L Boxes were not found upstream of other genes that have been determined to be RL activated in F. diplosiphon. One possibility is that they are regulated differently, since all seven average only two-fold RL induction (Stowe-Evans et al., 2004), making them much less light responsive than pcyA and cpc2.

The demonstration that the L Box has a key functional role in CCA regulation of pcyA expression (FIG. 12) correlates well with previous studies of the cpc2 promoter (Casey and Grossman, 1994). In that work, a deletion analysis of the cpc2 promoter revealed that CCA responsiveness was retained within a fragment containing the region from −87 to +14 (the numbering used here is based on the result presented in FIG. 10 and differs from the numbering used in Casey and Grossman, 1994, by −11 bp). Further deletion to −48, which truncated the promoter seven by upstream of the L Box, resulted in the loss of promoter activity. Because activity from the cpc2 promoter cannot be detected during GL growth, and the L Box is a RL activating element (FIG. 12), it is highly likely that the cpc2 promoter would be inactive under all light conditions if the L Box within it were mutated.

The presence of an L Box within the cpc2 promoter of the group III cyanobacterium Pseudanabaena sp. PCC 7409 (Dubbs and Bryant, 1993) suggests that a common signal transduction mechanism may control RL activated genes in at least some members of this group. The sequences of pcyA and cpeCDESTR in Pseudanabaena are not currently available so the presence of L Boxes in the promoters of these genes remains undetermined. CCA occurs in a large number of cyanobacterial species in a range of diverse habitats (Tandeau de Marsac, 1977). It will be interesting to determine how universally the L Box is used for transcriptional activation by RL in cyanobacteria.

In addition, the presence of an L Box within the GL up regulated cpeCDESTR promoter indicates that this element might function more broadly in the control of CCA. However, its location and orientation in that promoter (FIG. 11A) suggests that it has a different role than in the RL induced pcyA and cpc2 promoters. It is proposed that it is acting as a repressing element in the regulation of cpeCDESTR transcription during growth in RL, which, if true, would fit well with the recently recognized central role of this operon in up regulating the GL expressed cpeBA and pebAB operons through the production of CpeR (Cobley et al., 2002; Seib and Kehoe, 2002). It would also begin to provide a mechanism for how the Rca system could partially control the expression of GL activated genes during CCA, since previous molecular genetic work has established that the Rca system primarily contributes to the down regulation of such genes during RL growth (Seib and Kehoe, 2002; Alvey et al., 2003).

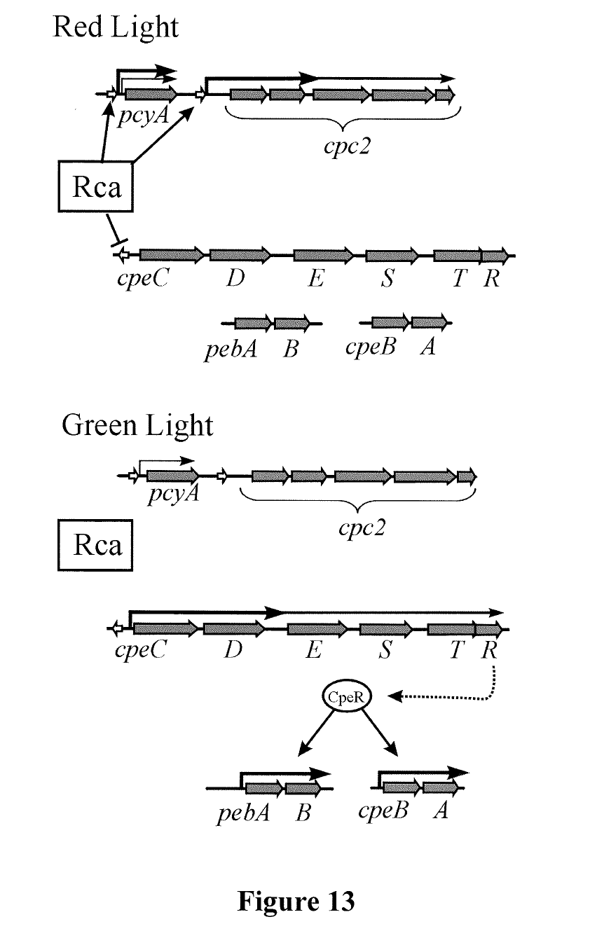

A model is therefore proposed in which the L Box is not only the binding site for a transcription factor that coordinates cpc2 and pcyA transcriptional activation in response to RL, but also the element that serves to coordinate the activation of these genes with the repression of transcription of cpeCDESTR and, thereby, cpeBA and pebAB (FIG. 13). More particularly, FIG. 13 shows a proposed model of how the Rca system regulates CCA controlled genes in F. diplosiphon. In RL, the Rca system (denoted by boxed Rca) directly or indirectly activates pcyA and cpc2 (operon is bracketed) transcription through the L Boxes (open arrows) in their promoters while repressing cpeCDESTR expression through the equivalent sequence within its promoter. The lack of CpeR production precludes transcription of pebAB. In GL, the Rca system does not activate the RL-specific transcription of either pcyA or cpc2, resulting in only low level expression of pcyA from its non-CCA responsive promoter. The absence of cpeCDESTR repression by the Rca system allows this operon to be expressed [in conjunction with the Cgi system (Kehoe and Gutu 2006), which is not included here], leading to CpeR (oval) production and, directly or indirectly, subsequent expression of pebAB and cpeBA. Bent arrows denote transcription (transcript accumulation for cpeCDESTR and pebAB); increasing arrow thicknesses correspond to higher levels. Straight arrows denote activation of gene expression and blocked line indicates repression of expression. Both types of activities may be direct or indirect. Dashed arrow denotes CpeR synthesis.

This model is currently being tested by mutation of the L Box within the cpeCDESTR promoter. Another issue still to be resolved is the identity of the transcription factor that binds the L Box. This protein has not yet been identified, but a prime candidate is RcaC for two reasons. First, it contains an OmpR/PhoB DNA binding domain which often bind direct repeat sequences of lengths similar to those in the L Box (Rampersaud et al., 1989). Second, the fact that an rcaC null mutant always produces PE, but never PC2, regardless of ambient light quality, is consistent with the proposed role of the L Boxes in the activation of RL induced operons and attenuation of GL induced operons (Chiang et al., 1992; Li and Kehoe, 2005). However, because no direct link has yet been provided between RcaC and the L Box it remains possible that RcaC acts indirectly, for example, by activating a gene encoding another transcription factor.

In this study, the L Box has been identified as the cis-acting element involved in the final step of a complex phosphorelay controlled by the prokaryotic phytochrome-class photoreceptor RcaE. Many prokaryotic phytochromes act through the phosphorylation of single domain response regulators similar to RcaF (Kehoe and Grossman, 1997; Yeh et al., 1997; Bhoo et al., 2001; Giraud et al., 2005) and thus are likely to function through complex phosphorelays as well. It will be interesting to uncover the extent to which direct repeats are used in bacteria as the final step in phytochrome-regulated phosphorelay systems. Such repeats may be especially important in cyanobacteria, where OmpR/PhoB superfamily components, which are prime candidates for interacting with such DNA elements, are an abundant class of two component proteins (50% of the total in S. elongatus PCC 7942, 32% of the total in N. punctiforme ATCC29133, and 36% of the total in A. variabilis ATCC29413) (http://genome.jgi-psf.org/mic_home.html).

METHODS: Strains and Growth Conditions—The wild type used here is a short-filament strain of F. diplosiphon designated Fd33 [previously SF33; (Cobley et al., 1993)]. The rcaE and rcaC null mutants were isolated from the wild type as described previously (Chiang et al., 1992; Kehoe and Grossman, 1996). All other mutants were isolated as pigmentation mutants from plates after heat-shock treatment, which mobilizes endogenous insertion elements in F. diplosiphon (K. Shockley, R. Alvey and D. Kehoe, unpublished results). All mutations were confirmed using PCR and sequencing. Strains were cultured as described previously (Seib and Kehoe, 2002).

PCR Amplification of pcyA: pcyA was originally PCR amplified from F. diplosiphon genomic DNA using standard methods and the following degenerate primers:

| | |
|---|---|
| pcyA1 | (5′ TGYTAYCARA CHCCHCARTT YCG 3′) |
| and | |
| pcyA2 | (5′ CGDGTYTTRT CRTTYTGYTG YTG 3′). |

Southern Blot, RNA Blot, and Phage Library Analyses: PCR amplification of F. diplosiphon genomic DNA was used to generate the pcyA probe that was used in these analyses. A PCR product of 404 by was amplified using two primers: fdpcya1 (5′ GTTTAGATAT TCTGCACTGT GTG 3′) and fdpcya2 (5′ TTTTGTACAG TAATTGCGCT GTC 3′). Radioactive labeling of this product with α³²P-dATP was performed as described previously (Seib and Kehoe, 2002). RNA extraction from cyanobacteria) cells and blot analyses were also conducted as previously described (Seib and Kehoe, 2002). For Southern blot analysis, genomic DNA isolated from wild type F. diplosiphon was digested with Apo I, Hae II, and Hpa I and electrophoretically separated on a 0.7% agarose TAE gel. Transfer of the digested DNA was performed as with RNA sample for the RNA blot analysis, except that the gel was pretreated in once in 0.25M HCl for 15 min, then twice in 0.5 M NaOH/1M NaCl for 15 min each, then twice in 1M Tris (pH 7.5)/3M NaCl for 15 min each. Hybridization of the labeled probe was also conducted as described for the RNA blot analysis with the exception of the blot washes, which were performed at progressively higher temperatures up to 45° C. A phage library was constructed using the Novagen BlueSTAR Vector system and F. diplosiphon DNA, and screened using standard techniques (Sambrook et al., 1989).

Construction of pcyA Expression Vector: The pcyA gene from F. diplosiphon was PCR amplified from chromosomal DNA using the following primers, which contained the indicated/underlined restriction sites: FdpcyAEcoRIfwd 5′ GGAATTCATC TTGAAGTCTA TGGAAGCGTC G 3′ and FdpcyAHindIIIrev 5′ CCCAAGCTTT TAAGCTACAT AGTCAAAAAG CATAG 3′. The PCR product was then cut with the EcoR I and Hind III and inserted into similarly cleaved pMal-c2 (NEB, Ipswich, Mass.) to yield pMal_pcyA. The integrity of the plasmid constructs were verified by complete DNA sequencing of the insert (Seqlab, Göttingen, Germany). The construct placed the pcyA gene downstream of and in-frame with the maltose-binding protein gene of E. coli, under the control of a Ptac promoter. A recognition sequence for Factor Xa protease is located upstream of the cloned gene.

Expression and Partial Purification of Recombinant PcyA: The E. coli strain JM109 containing pMal_pcyA was grown at 37° C. in 500 ml batches of Luria-Bertani media containing ampicillin (100 μg/ml) to an OD_{578 nm} of 0.6. Expression of pcyA was induced by the addition of 1 mM isopropylthio-β-galactoside (IPTG) to the cultures and incubation for an additional 3 h. Bacteria were then harvested by centrifugation and the bacterial pellet from each one liter of culture was resuspended in 8 ml of lysis buffer (50 mM Tris-HCl (pH 8.0)/100 mM NaCl/0.05% Triton X-100) and disrupted three times with a French pressure cell (20,000 lb/in.²). Cell debris was removed by centrifugation for 30 min at 100,000×g. The resulting supernatant was used for enzymatic assays.

Standard Bilin Reductase Activity and Cph1 Attachment Assays: Assays for bilin reductase activity were performed as described previously (Kohchi et al., 2001). To test for PCB chromophore attachment and function, recombinant Cph1 apo-phytochrome was added to the bilin reaction mixture, incubated for another 30 min at RT under green safe light, and the difference spectrum was measured as previously described (Yeh et al., 1997).

Transcription Start Site Determinations: Transcription start sites were identified using ribonuclease protection (for pcyA) and primer extension (for cpc2) assays. A region of the pcyA promoter and coding sequence was PCR amplified using primers fcpcya2 and fdpcya3 (5′ GACGAGGCTT GCGGAGTGG 3′). The product was cut with Xba I and the 685 bp product was isolated and cloned into pBluescript SK+II from Stratagene (San Diego, Calif.). The sequence was verified and the plasmid, ppcyAPE, was used to synthesize radiolabeled RNA. The plasmid was cut with BamH I and the Riboprobe in vitro transcription kit from Promega (Madison, Wis.) was used to synthesize RNA, using T3 RNA polymerase and α³²P-CTP from GE Biosciences (Piscataway, N.J.). The resulting radiolabeled RNA was used in a reaction using the Ribonuclease Protection Assay (RPA III) kit from Ambion (Austin, Tex.). Reactions were carried out according to the manufacturer's instructions. RNA samples were obtained from RL and GL grown cultures using previously described methods (Seib and Kehoe, 2002). Assay products were run on a 5% sequencing gel at 55 W for 2 h, along with a sequencing reaction products generated using the same plasmid, the primer pcyARPAseq (5′ TCTAGATGCA GTTTGCGAAA TTG 3′), and the Sequenase Version 2.0 DNA Sequencing Kit from USB (Cleveland, Ohio).

A primer extension assay was used to determine the transcription start site of cpc2. The primer cpc2PE (5′ GCCCACAAGC CTAGATTAAG AA 3′) was used along with the primer extension system from Promega (Madison, Wis.). Reactions were performed according to the instructions provided, using the optional denaturing hybridization solution. Sequencing reaction products were generated using the same primer and the USB kit described above. RNA samples were obtained from RL and GL grown cultures using previously described methods (Seib and Kehoe, 2002).

GUS Assays: Three pcyA:gusA constructs were created. All fusions of the pcyA promoter to the E. coli gusA gene were made using PCR amplification and the primers pcyAGUS1 (5′ gcggcatgcA CAAACCACAG AAATGCCTGA G 3′) and newpcyAGUS2 (5′ gcgggatccC ATAGACTTCA ACATACGCTT CTCTTTGAGT ATAGGATTAA C 3′). The lower case letters in these primers denote added sequence, including restriction endonuclease sites. The mutation in the STRR5 repeats within the pcyA promoter was made using a two step process. First, two PCR amplification reactions were performed using primers RBKOL2B (5′ GCGCTCGAGA TGCATGTATC CAAAGTTA 3′) and pcyAGUS1 for one reaction and RBKO2RB (5′ GCGCTCGAGA TGCATGTATC CAATTTAT 3′) and newpcyAGUS2 for the other. The PCR products of these two reactions were then mixed and used as the template for a third PCR amplification reaction using pcyAGUS1 and newpcyAGUS2. The L Box was mutated using via PCR amplification and primers pcyAGUS1 and pcyAMGP (5′ gcgggatccC ATAGACTTCA ACATACGCTT CTCTTTGAGT ATAGGATTAA CCTACCTTGG CGTATAGCCG TAGACTTATC GTACGTATTT GCAGATT 3′). The lower case letters in these primers denote added sequence, including restriction endonuclease sites, while underlined sequences are sites of L Box mutation changes. The three final one kbp PCR products were cleaved with BamH I and Sph I and inserted into similarly cut p2.7GI (Casey and Grossman, 1994) to make plasmids ppcyApGUS, ppcyA-LGUS, and ppcyA-SGUS. All PCR amplified DNA regions and ligation junctions were sequenced to insure that no mutations had occurred during clone synthesis. Transformations of F. diplosiphon with these plasmids were conducted using previously described methods (Kehoe and Grossman, 1998).

GUS enzyme assay methods were modified from previous protocols (Jefferson et al., 1986; Casey and Grossman, 1994). Transformed F. diplosiphon cells were grown in 50 ml BG-11 cultures supplemented with kanamycin (10 μg/ml) to an A₇₅₀ of 0.7 in 15 μmol of either RL or GL m⁻²s⁻¹. Then 200 μl of each culture was transferred to a 1.5 ml microfuge tube and centrifuged for 5 min at 12,000×g at RT. The pellets were washed in 1 ml of GUS assay buffer (50 mM NaPO₄/1 mM EDTA) with chloramphenicol (6.25 μg/ml), then resuspended in 1 ml GUS assay buffer. Cells were permeabilized by the addition of 2 drops of 0.1% SDS and 4 drops chloroform, followed by vortexing on the maximum setting for 10 s. Assays were carried out in 96 well microliter plates. Reactions were performed using 175 μl of GUS assay buffer containing 1.25 mM 4-Nitrophenyl β-D-glucuronide. Aliquots of 25 μl of lysate were added to each well using a multi-channel pipetter. A reading was taken every 4 min for 60 min at 405 nm using a BIO-TEK Synergy HT spectrophotometer. GUS activity was calculated by measuring the absorbance of each sample. Activity was quantified as nmol of product per mg of protein per min. Protein concentrations were determined using the Pierce (Rockford, Ill.) BCA protein assay reagent kit.

Genome Analysis: Sequence alignments were made using CLUSTALW at the San Diego Supercomputer Center Biology WorkBench website (http://workbench.sdsc.edu/) with minor adjustments using protein Basic Local Alignment Search Tool (BLAST) analysis information (Altschul et al., 1990) (http://www.ncbi.nlm.nih.gov/BLAST). Accession numbers for the sequences compared to F. diplosiphon PcyA are Anabaena sp. PCC 7120 pcyA: AAK38587, Synechocystis sp. PCC 6803 pcyA: BAA10653, and Pseudanabaena sp. PCC 7409 cpc2: M99427.

The DNA sequence of the entire ca. 4.3 kbp region of DNA located between apc and cpc2 sequences and the newly acquired DNA sequence 5′ of cpeC has been placed in a public repository. Sequence data from this article can be found in the EMBL/GenBank data libraries.

Future work is dedicated to transferring genes encoding the Rca system components, along with the cpc2 promoter, from a cyanobacterium to E. coli. The result will be an E. coli strain capable of using externally supplied light color to tightly regulate the transcription, in vivo, of any gene that has been placed under the control of the cpc2 promoter.

The pathway to be transferred is the “Rca” pathway, which contains three regulatory proteins, RcaE, RcaF, and RcaC, that together make up a type of prokaryotic signal transduction pathway called a phosphorelay. Such pathways allow bacteria to sense and respond to different environmental cues. Their activity is regulated by the reversible phosphorylation of specific aspartate and histidine residues within the regulatory proteins. Sensors detect signals, and response regulators transduce signals as phosphorylation events, eliciting cellular responses.

Although the signal(s) that control most bacterial phosphorelays are unknown, they are well known for the Rca system: it is activated by red light and inactivated by green light. RcaE, the photoreceptor, is activated by red light through a covalently linked open chain tetrapyrrole chromophore (bilin), which isomerizes upon photon absorption. RcaE contains a bilin binding domain and an autophosphorylation domain. In phosphorelays, signal propagation is initiated by autophosphorylation at a histidine residue within such domains. For RcaE, this apparently occurs in red light and does not occur in green light. RcaF is a “response regulator” protein. These accept phosphoryl groups from the histidine and pass them on to a third protein, which is RcaC in this system. RcaC is also a response regulator, but it contains a PhoB/OmpR class DNA binding domain. RcaC abundance is six-fold higher in red light than green light.

The Rca system has been shown to be exclusively responsible for controlling a number of genes' high level induction in red light and silencing in green light. The best studied are cpc2 and pcyA. cpc2 encodes red light absorbing proteins found in the organism's photosynthetic light harvesting antennae. Because they make up more than 30% of the total soluble protein in these cells, this operon is very highly transcribed in red light. pcyA is much less highly induced and encodes the enzyme needed for synthesis of the red light absorbing bilins used in the antennae.

The promoters of both cpc2 and pcyA contain an “L Box”, which is a 7 by sequence that is repeated once, with the repeats separated by 4 bp. The L Box is centered at −35 relative to the transcription start site in both promoters. It has been shown that this DNA sequence confers red light inducibility to these promoters and that it is bound by RcaC (Li, Alvey, and Kehoe, unpublished). Thus, the Rca pathway has been characterized from photoreceptor to transcription factor and can now transfer this system into E. coli, a commercially useful species.

In order to achieve this goal, there are a number of technical obstacles that will need to be overcome, and several additional issues that will likely need to be addressed (below) in order to make this invention work effectively.

1. Production of the photoreceptor bilin. RcaE-class photoreceptors require a covalently attached, heme-derived bilin, usually phycocyanobilin (PCB), to function properly. E. coli cells produce enough heme so that if the enzymes heme oxygenase (encoded by ho1 in cyanobacteria) and phycocyanobilin oxidoreductase (encoded by pcyA) are provided, sufficient PCB is produced to assemble functional photoreceptors in vivo (3). Thus, ho1 and pcyA will need to be inserted into E. coli under the control of a weak, constitutive promoter. Currently, the present inventors have made this plasmid and transformed it into E. coli.

2. Use of Synechococcus sp. PCC7335 RcaE or replacement of the F. diplosiphon RcaE bilin binding domain. Unlike most related photoreceptors, F. diplosiphon RcaE does not autocatalytically attach PCB in E. coli cells engineered to produce it. Therefore, either the closely related RcaE from Synechococcus sp. PCC7335 will be used, or if this protein also does not attach a chromophore autocatalytically, the F. diplosiphon RcaE chromophore binding domain will be replaced by equivalent domains from two closely related photoreceptors PixJ1 and Cph1, both of which attach PCB in E. coli. The atomic structure of these domain are known and these substitutions are highly likely to be functional. These will be made in any case, because they provide an additional benefit of making three different color responsive systems possible, since RcaE is red/green photoreversible, PixJ1 is blue/green photoreversible, and Cph1 is red/far red photoreversible. These chimaeric constructs are not likely to affect RcaE-RcaF signal transduction, since sensor-response regulator interactions involve only the autophosphorylation domain.

3. RcaC level adjustments in red and green light. This system will likely need to be fine tuned for optimal performance in E. coli. In cyanobacteria, the six-fold change in RcaC abundance in red versus green light helps fully shut off cpc2 transcription in green light and may help fully induce it in red light. These abundance changes will be mirrored in E. coli by placing rcaC under the control of the pcyA gene promoter, which is five fold up regulated by the Rca system but expressed at a low level in green light. This will create a positive feedback loop, making RcaC more abundant when the system is activated and less abundant when inactivated. Some promoter strength adjustments may be required, and the RcaC degradation rate must be high enough to rapidly remove most RcaC after a switch to the inactive state (some must be present to allow reactivation). This can be achieved by adding a short cleavage recognition peptide to RcaC.

4. Making the cpc2 promoter functional in E. coli. It is not yet clear how the cpc2 promoter, with RcaC present, will function in E. coli. While the basal transcriptional machinery acts similarly in E. coli and cyanobacteria, the features of the regulatory components are often different and thus the cpc2 promoter, and perhaps RcaC, may require adjustments to function optimally in E. coli. If cpc2 does not function in E. coli, it will be tested whether the problem is the sequence controlling the basal expression of this promoter by replacing the “Pho Box”, which is similar in position and structure (but not sequence) to the L Box in E. coli PhoB regulated promoters, with an L Box. Alternatively, suboptimal activity could be due to incorrect interactions between RcaC and holo-RNA polymerase. PhoB/OmpR class transcription factors interact with holo-RNA polymerase differently. OmpR interacts with the α subunit, but PhoB interacts with the σ70 subunit. However, both of these interact with RNA polymerase through the “turn” within their helix-turn-helix DNA binding motif. If there are interaction problems between RcaC and holo-RNA polymerase, the turns of the helix-turn-helix DNA binding motifs from both PhoB and OmpR will be substituted for the comparable turn within RcaC.

This invention will provide a prokaryotic regulative promoter that is fully off in non-inducing conditions and highly expressed when induced. The inducer, visible light, can be instantaneously applied and removed in very specific intensities and color ratios, allowing highly tunable expression of target genes. These features are significant improvements over currently available inducible promoter systems for at least the following reasons. (1) Those that are highly inducible (such as the lac system) cannot be completely shut off, a problem when the induced protein affects cell physiology or is toxic in small amounts. (2) Those that can be completely shut off (such as the arabinose system) can only be weakly induced, which often provides insufficient amounts of protein product. (3) No commercially available prokaryotic system is capable of incrementally activating and inactivating a promoter by changing the ratio of two wavelengths of light provided. (4) Many chemicals and treatments currently used as inducing agents alter E. coli physiology. Sugars such as lactose, arabinose, and rhamnose have pleiotropic effects and treatments such as cold or heat shock, or exposure to high metal concentrations (such as copper) also alter the physiology of these cells. Except for DNA damage repair, visible light is not used or sensed by E. coli and thus its application does not effect these cells or alter the expression of any other genes in this organism.

While an exemplary embodiment incorporating the principles of the present invention has been disclosed hereinabove, the present invention is not limited to the disclosed embodiments. Instead, this application is intended to cover any variations, uses, or adaptations of the invention using its general principles of utilizing a prokaryotic phytochrome or a chimera of such a photoreceptor and its, or another, downstream regulatory system for controlling gene expression. Further, this application is intended to cover such departures from the present disclosure as come within known or customary practice in the art to which this invention pertains.

### REFERENCES

The following references are incorporated herein by reference in their entirety:
1. Alvey, R. M., Frankenberg-Dinkel, N., and Kehoe, D. M. 2006. The cyanobacterial phytochrome-class photoreceptor RcaE regulates the phycocyanobilin oxidoreductase gene pcyA via a direct repeat DNA sequence. Plant Cell submitted;
2. Baneyx, F. 1999. Recombinant protein expression in Escherichia coli. Curr. Opin. Biotech. 10:411-421;
3. Bennett, A., and Bogorad, L. 1973. Complementary chromatic adaptation in a filamentous blue-green alga. J. Cell. Biol. 58:419-435;
4. Chiang, G. G., Schaefer, M. R., and Grossman, A. R. 1992. Complementation of a red-light-indifferent cyanobacterial mutant. Proc. Natl. Acad. Sci. USA 89: 9415-9419;
5. Gambetta, G. A., and Lagarias, J. C. 2001. Genetic engineering of phytochrome biosynthesis in bacteria. Proc. Natl. Acad. Sci. USA 98:10566-10571;
6. Giacalone, M. J., et al. 2006. Toxic protein expression in Escherichia coli using a rhamnose-based tightly regulated and tunable promoter system. BioTechniques 40:355-364;
7. Kaern, M., Blake, W. J., and Collins, J. J. 2003. The engineering of gene regulatory networks. Annu. Rev. Biomed. Eng. 5:179-206;
8. Kehoe, D. M., and Grossman, A. R. 1996. Similarity of a chromatic adaptation sensor to phytochrome and ethylene receptors. Science 273:1409-1412;
9. Kehoe, D. M., and Grossman, A. R. 1997. New classes of mutants in complementary chromatic adaptation provide evidence for a novel four-step phosphorelay system. J. Bacteria 179:3914-3921;
10. Li, L., and Kehoe, D. M. 2005. In vivo analysis of the roles of conserved aspartate and histidine residues within a complex response regulator. Mol. Microbiol. 55:1538-1552;
11. Shibato, J., Agrawal, G. K., Kato, H., and Asayama, M. 2002. The 5′ upstream cis-acting sequences of a cyanobacterial psbA gene: analysis of their roles in basal, light-dependent and circadian transcription. Mol. Gen. Genom. 267:684-694;
12. Swartz, J. R. 2001. Advances in Escherichia coli production of therapeutic proteins. Curr. Opin. Biotech. 12:195-201;
13. Terauchi, K., Montgomery, B. L., Grossman, A. R., Lagarias, J. C., and Kehoe, D. M. 2004. RcaE is a complementary chromatic adaptation photoreceptor required for green and red light responsiveness. Mol. Microbiol. 51:567-77;
14. Yoshihara, S., Shimada, T., Matsuoka, D., Zikihara, K., Kohchi, T., and Tokutomi, S. 2006. Reconstitution of blue-green reversible photoconversion of a cyanobacterial photoreceptor, PixJ1, in phycocyanobilin-producing Escherichia coli. Biochemistry 45:3775-84;
15. Wagner, J. R., Brunzelle, J. S., Forest, K. T., Vierstra, R. D. 2005. A light-sensing knot revealed by the structure of the chromophore-binding domain of phytochrome. Nature 438: 325-331;
16. Oelmuller, R., Grossman, A. R., Briggs, W. R. “PHOTOREVERSIBILITY OF THE EFFECT OF RED AND GREEN LIGHT-PULSES ON THE ACCUMULATION IN DARKNESS OF MESSENGER-RNAS CODING FOR PHYCOCYANIN AND PHYCOERYTHRIN IN FREMYELLA-DIPLOSIPHON.” Plant Physiology 88 1084-1091, 1988;
17. Altschul, S. F., Gish, W., Miller, W., Myers, E. W., and Lipman, D. J. (1990). Basic local alignment search tool. J. Mol. Biol. 215, 403-410;
18. Alvey, R. M., Karty, J. A., Roos, E., Reilly, J. P., and Kehoe, D. M. (2003). Lesions in phycoerythrin chromophore biosynthesis in Fremyella diplosiphon reveal coordinated light regulation of apoprotein and pigment biosynthetic enzyme gene expression. Plant Cell 15, 2448-2463;
19. Anderson, L. K., and Grossman, A. R. (1990). Genes for phycocyanin subunits in Synechocystis sp. strain PCC 6701 and assembly mutant UV16. J. Bacterial. 172, 1289-1296;
20. Anderson, L. K., and Toole, C. M. (1998). A model for early events in the assembly pathway of cyanobacterial phycobilisomes. Mol. Microbiol. 30, 467-474;
21. Angeloni, S. V., and Potts, M. (1994). Analysis of the sequences within and flanking the cyanoglobin-encoding gene, glbN, of the cyanobacterium Nostoc commune UTEX 584. Gene 146, 133-134;
22. Beale, S. I. (1993). Biosynthesis of phycobilins. Chem. Rev. 93, 785-802;
23. Bhaya, D., Takahashi, A., and Grossman, A. R. (2001). Light regulation of type IV pilus-dependent motility by chemosensor-like elements in Synechocystis PCC6803. Proc. Natl. Acad. Sci. USA 98, 7540-7545;
24. Bhoo, S. H., Davis, S. J., Walker, J., Karniol, B., and Vierstra, R. D. (2001). Bacteriophytochromes are photochromic histidine kinases using a biliverdin chromophore. Nature 414, 776-779;
25. Bogorad, L. (1975). Phycobiliproteins and complementary chromatic adaptation. Ann. Rev. Plant Physiol. 26, 369-401;
26. Bryant, D. A. (1981). The photoregulated expression of multiple phycocyanin species: a general mechanism for the control of phycocyanin synthesis in chromatically adapting cyanobacteria. Eur. J. Biochem. 119, 425-429;
27. Casey, E. S., and Grossman, A. (1994). In vivo and in vitro characterization of the light-regulated cpcB2A2 promoter of Fremyella diplosiphon. J. Bacteriol. 176, 6362-6374;
28. Chapman, D. J., Cole, W. J., and Siegelman, H. W. (1967). Structure of phycoerythrobilin. J. Am. Chem. Soc. 89, 5976-5977;
29. Cobley, J. G., Zerweck, E., Reyes, R., Mody, A., Seludo-Unson, J. R., Jaeger, H., Weerasuriya, S., and Navankasattusas, S. (1993). Construction of shuttle plasmids which can be efficiently mobilized from Escherichia coli into the chromatically adapting cyanobacterium, Fremyella diplosiphon. Plasmid 30, 90-105;
30. Cobley, J. G., Clark, A. C., Weerasurya, S., Queseda, F. A., Xiao, J. Y., Bandrapali, N., D'Silva, I., Thounaojam, M., Oda, J. F., Sumiyoshi, T., and Chu, M. H. (2002). CpeR is an activator required for expression of the phycoerythrin operon (cpeBA) in the cyanobacterium Fremyella diplosiphon and is encoded in the phycoerythrin linker-polypeptide operon (cpeCDESTR). Mol. Microbiol. 44, 1517-1531;
31. Cole, W. J., Chapman, D. J., and Siegelman, H. W. (1967). The structure of phycocyanobilin. J. Am. Chem. Soc. 89, 3642;
32. CConley, P. B., Lemaux, P. G., and Grossman, A. R. (1985), Cyanobacterial light-harvesting complex subunits encoded in two red-light induced transcripts. Science 230, 550-553;
33. Conley, P. B., Lemaux, P. G., and Grossman, A. (1988). Molecular characterization and evolution of sequences encoding light-harvesting components in the chromatically adapting cyanobacterium Fremyella diplosiphon. J. Mol. Biol. 199, 447-465;
34. Conley, P. B., Lemaux, P. G., Lomax, T. L., and Grossman, A. R. (1986). Genes encoding major light-harvesting polypeptides are clustered on the genome of the cyanobacterium Fremyella diplosiphon. Proc. Natl. Acad. Sci. USA 83, 3924-3928;
35. Cornejo, J., and Beale, S. I. (1997). Phycobilin biosynthetic reactions in extracts of cyanobacteria. Photosynth. Res. 51, 223-230;
36. Cornejo, J., Willows, R. D., and Beale, S. I. (1998). Phytobilin biosynthesis: cloning and expression of a gene encoding soluble ferredoxin-dependent heme oxygenase from Synechocystis sp. PCC 6803. Plant J. 15, 99-107;
37. Crespi, H. L., Boucher, L. J., Norman, G. D., Katz, J. J., and Dougherty, R. C. (1967). Structure of phycocyanobilin. J. Am. Chem. Soc. 89, 3642-3643;
38. Dammeyer, T., and Frankenberg-Dinkel, N. (2006). Insights into phycoerythrobilin biosynthesis point towards metabolic channeling. J Biol Chem in press;
39. Davis, S. J., Vener, A. V., and Vierstra, R. D. (1999a). Bacteriophytochromes: phytochrome-like photoreceptors from nonphotosynthetic eubacteria. Science 286, 2517-2520;
40. Davis, S. J., Kurepa, J., and Vierstra, R. D. (1999b). The Arabidopsis thaliana HY1 locus, required for phytochrome-chromophore biosynthesis, encodes a protein related to heme oxygenases. Proc. Natl. Acad. Sci. USA 96, 6541-6546;
41. Dubbs, J. M., and Bryant, D. A. (1993). Organization and transcription of the genes encoding two differentially expressed phycocyanins in the cyanobacterium Pseudanabaena sp PCC 7409. Photosynth. Res. 36, 169-183;
42. Federspiel, N. A., and Grossman, A. R. (1990). Characterization of the light-regulated operon encoding the phycoerythrin-associated linker proteins from the cyanobacterium Fremyella diplosiphon. J. Bacteriol. 172, 4072-4081;
43. Federspiel, N. A., and Scott, L. (1992). Characterization of a light-regulated gene encoding a new phycoerythrin-associated linker protein from the cyanobacterium Fremyella diplosiphon. J. Bacteriol. 179, 5994-5998;
44. Fiedler, B., Borner, T., and Wilde, A. (2005). Phototaxis in the cyanobacterium Synechocystis sp. PCC 6803: role of different photoreceptors. Photochem. Photobiol. 81, 1481-1488;
45. Fiedler, B., Broc, D., Schubert, H., Rediger, A., Borner, T., and Wilde, A. (2004). Involvement of cyanobacterial phytochromes in growth under different light qualities and quantities. Photochem. Photobiol. 79, 551-555;
46. Frankenberg, N., and Lagarias, J. C. (2003). Phycocyanobilin:ferredoxin oxidoreductase of Anabaena sp PCC 7120-Biochemical and spectroscopic characterization. J. Biol. Chem. 278, 9219-9226;
47. Frankenberg, N., Mukougawa, K., Kohchi, T., and Lagarias, J. C. (2001). Functional genomic analysis of the HY2 family of ferredoxin-dependent bilin reductases from oxygenic photosynthetic organisms. Plant Cell 13, 965-978;
48. Giraud, E., Fardoux, L., Fourrier, N., Hannibal, L., Genty, B., Bouyer, P., Dreyfus, B., and Vermeglio, A. (2002). Bacteriophytochrome controls photosystem synthesis in anoxygenic bacteria. Nature 417, 202-205;
49. Giraud, E., Zappa, S., Vuillet, L., Adriano, J. M., Hannibal, L., Fardoux, J., Berthomieu, C., Bouyer, P., Pignol, D., and Vermeglio, A. (2005). A new type of bacteriophytochrome acts in tandem with a classical bacteriophytochrome to control the antennae synthesis in Rhodopseudomonas palustris. J. Biol. Chem. 280, 32389-32397;
50. Glauser, M., Sidler, W. A., Graham, K. W., Bryant, D. A., Frank, G., Wehrli, E., and Zuber, H. (1992). Three C-phycoerythrin-associated linker polypeptides in the phycobilisome of green-light-grown Calothrix sp. PCC 7601 (cyanobacteria). FEBS Lett. 297, 19-23;
51. Glazer, A. N. (1989). Light guides. Directional energy transfer in a photosynthetic antenna. J. Biol. Chem. 264, 1-4;
52. Grossman, A. R. (2003). A molecular understanding of complementary chromatic adaptation. Photosynth. Res. 76, 207-215;
53. Hagiwara, Y., Sugishima, M., Takahashi, Y., and Fukuyama, K. (2006a). Induced-fitting and electrostatic potential change of PcyA upon substrate binding demonstrated by the crystal structure of the substrate-free form. FEBS Lett. 580, 3823-3828;
54. Hagiwara, Y., Sugishima, M., Takahashi, Y., and Fukuyama, K. (2006b). Crystal structure of phycocyanobilin:ferredoxin oxidoreductase in complex with biliverdin IX alpha, a key enzyme in the biosynthesis of phycocyanobilin. Proc. Natl. Acad. Sci. USA 103, 27-32;
55. Hubschmann, T., Yamamoto, H., Gieler, T., Murata, N., and Borner, T. (2005). Red and far-red light alter the transcript profile in the cyanobacterium Synechocystis sp PCC 6803: Impact of cyanobacterial phytochromes. FEBS Lett. 579, 1613-1618;
56. Jaubert, M., Zappa, S., Fardoux, J., Adriano, J. M., Hannibal, L., Elsen, S., Layergne, J., Vermeglio, A., Giraud, E., and Pignol, D. (2004). Light and redox control of photosynthesis gene expression in Bradyrhizobium-Dual roles of two PpsR. J. Biol. Chem. 279, 44407-44416;
57. Jefferson, R. A., Burgess, S. M., and Hirsh, D. (1986). β-Glucuronidase from Escherichia coli as a gene-fusion marker. Proc. Natl. Acad. Sci. USA 83, 8447-8451;
58. Jiang, Z., Swem, L. R., Rushing, B. G., Devanathan, S., Tollin, G., and Bauer, C. E. (1999). Bacterial photoreceptor with similarity to photoactive yellow protein and plant phytochromes. Science 285, 406-409;
59. Karniol, B., Wagner, J. R., Walker, J. M., and Vierstra, R. D. (2005). Phylogenetic analysis of the phytochrome superfamily reveals distinct microbial subfamilies of photoreceptors. Biochem J. 392, 103-116;
60. Katayama, M., Xiao, X. G., Kobayashi, M., Kanehisa, M., and Ikeuchi, M. (2004). Light-responsive genes that are regulated by phytochrome-like proteins in Synechocystis sp. PCC6803. Plant Cell Physiol. 45, s124;
61. Kehoe, D. M., and Grossman, A. R. (1998). Use of molecular genetics to investigate complementary chromatic adaptation: advances in transformation and complementation. Methods Enzymol. 297, 279-290;
62. Kehoe, D. M., and Gutu, A. (2006). Responding to color: the regulation of complementary chromatic adaptation. Annu. Rev. Plant Biol. 57, 127-150;
63. Kohchi, T., Mukougawa, K., Frankenberg, N., Masuda, M., Yokota, A., and Lagarias, J. C. (2001). The Arabidopsis HY2 gene encodes phytochromobilin synthase, a ferredoxin-dependent biliverdin reductase. Plant Cell 13, 425-436;
64. Liotenberg, S., Campbell, D., Rippka, R., Houmard, J., and Tandeau de Marsac, N. (1996). Effect of the nitrogen source on phycobiliprotein synthesis and cell reserves in a chromatically adapting filamentous cyanobacterium. Microbiology 142, 611-622;
65. Lomax, T. L., Conley, P. B., Schilling, J., and Grossman, A. R. (1987). Isolation and characterization of light-regulated phycobilisome linker polypeptide genes and their transcription as a polycistronic mRNA. J. Bacteriol. 169, 2675-2684;
66. MacColl, R. (1998). Cyanobacterial phycobilisomes. J. Struct. Biol. 124, 311-334;
67. Mazel, D., Houmard, J., Castets, A. M., and Tandeau de Marsac, N. (1990). Highly repetitive DNA sequences in cyanobacterial genomes. J. Bacteriol. 172, 2755-2761;
68. Mazel, D., Guglielmi, G., Houmard, J., Sidler, W., Bryant, D. A., and Tandeau de Marsac, N. (1986). Green light induces transcription of the phycoerythrin operon in the cyanobacterium Calothrix 7601. Nucleic Acids Res. 14, 8279-8290;
69. Meeks, J. C., Elhai, J., Thiel, T., Potts, M., Larimer, F., Lamerdin, J., Predki, P., and Atlas, R. (2001). An overview of the genome of Nostoc punctiforme, a multicellular, symbiotic cyanobacterium. Photosynth. Res. 70, 85-106;
70. Montgomery, B. L., and Lagarias, J. C. (2002). Phytochrome ancestry: sensors of bilins and light. Trends Plant Sci. 7, 357-366;
71. Muramoto, T., Kohchi, T., Yokota, A., Hwang, I., and Goodman, H. M. (1999). The Arabidopsis photomorphogenic mutant hy1 is deficient in phytochrome chromophore biosynthesis as a result of a mutation in a plastid heme oxygenase. Plant Cell 11, 335-348;
72. Ng, W. O., Grossman, A. R., and Bhaya, D. (2003). Multiple light inputs control phototaxis in Synechocystis sp. strain PCC6803. J. Bacteriol. 185, 1599-1607;
73. Ó Carra, P., Ó Heocha, C., and Carroll, D. M. (1964). Spectral properties of phycobilins. 2. Phycoerythrobilin. Biochemistry 3, 1343-1350;
74. Ó Heocha, C. (1963). Spectral properties of the phycobilins. I. Phycocyanobilin. Biochemistry 2, 375-382;
75. Oberpichler, I., Molina, I., Neubauer, O., and Lamparter, T. (2006). Phytochromes from Agrobacterium tumefaciens: difference spectroscopy with extracts of wild type and knockout mutants. FEBS Lett. 580, 437-442;
76. Oelmüller, R., Conley, P. B., Federspiel, N., Briggs, W. R., and Grossman, A. R. (1988). Changes in accumulation and synthesis of transcripts encoding phycobilisome components during acclimation of Fremyella diplosiphon to different light qualities. Plant Physiol. 88, 1077-1083;
77. Ohmori, M., Terauchi, K., Okamoto, S., and Watanabe, M. (2002). Regulation of cAMP-mediated photosignaling by a phytochrome in the cyanobacterium Anabaena cylindrica. Photochem. Photobiol. 75, 675-679;
78. Rampersaud, A., Norioka, S., and Inouye, M. (1989). Characterization of OmpR binding sequences in the upstream region of the ompF promoter essential for transcriptional activation. J. Biol. Chem. 264, 18693-18700;
79. Sambrook, J., Fritsch, E. F., and Maniatis, T. (1989). Molecular cloning: a laboratory manual. (Cold Spring Harbor, N.Y.: Cold Spring Harbor Laboratory Press);
80. Schmidt-Goff, C. M., and Federspiel, N. A. (1993). in vivo and in vitro footprinting of a light-regulated promoter in the cyanobacterium Fremyella diplosiphon. J. Bacteriol. 175, 1806-1813;
81. Schmitz, O., Katayama, M., Williams, S. B., Kondo, T., and Golden, S. S. (2000). CikA, a bacteriophytochrome that resets the cyanobacterial circadian clock. Science 289, 765-768;
82. Seib, L. O., and Kehoe, D. M. (2002). A turquoise mutant genetically separates expression of genes encoding phycoerythrin and its associated linker peptides. J. Bacteriol. 184, 962-970;
83. Sobczyk, A., Schyns, G., Tandeau de Marsac, N., and Houmard, J. (1993). Transduction of the light signal during complementary chromatic adaptation in the cyanobacterium Calothrix sp. PCC 7601: DNA-binding proteins and modulation by phosphorylation. EMBO J. 12, 997-1004;
84. Sobczyk, A., Bely, A., Tandeau de Marsac, N., and Houmard, J. (1994). A phosphorylated DNA-binding protein is specific for the red-light signal during complementary chromatic adaptation in cyanobacteria. Mol. Microbiol. 13, 875-885;
85. Stowe-Evans, E. L., Ford, J., and Kehoe, D. M. (2004). Genomic DNA microarray analysis: identification of new genes regulated by light color in the cyanobacterium Fremyella diplosiphon. J. Bacteriol. 186, 4338-4349;
86. Tandeau de Marsac, N. (1977). Occurrence and nature of chromatic adaptation in cyanobacteria. J. Bacteriol. 130, 82-91;
87. Tandeau de Marsac, N. (1983). Phycobilisomes and complementary chromatic adaptation in cyanobacteria. Bull. Inst. Pasteur 81, 201-254;
88. Toole, C. M., Plank, T. L., Grossman, A. R., and Anderson, L. K. (1998). Bilin deletions and subunit stability in cyanobacterial light-harvesting proteins. Mol. Microbiol. 30, 475-486;
89. Troxler, R. F. (1972). Synthesis of bile pigments in plants. Formation of carbon monoxide and phycocyanobilin in wild-type and mutant strains of the alga, Cyanidium caldarium. Biochemistry 11, 4235-4242;
90. Tu, S. L., Sughrue, W., Britt, R. D., and Lagarias, J. C. (2006). A conserved histidine-aspartate pair is required for exovinyl reduction of biliverdin by a cyanobacterial phycocyanobilin:ferredoxin oxidoreductase. J. Biol. Chem. 281, 3127-3136;
91. Tu, S. L., Gunn, A., Toney, M. D., Britt, R. D., and Lagarias, J. C. (2004). Biliverdin reduction by cyanobacterial phycocyanobilin:ferredoxin oxidoreductase (PcyA) proceeds via linear tetrapyrrole radical intermediates. J. Am. Chem. Soc. 126, 8682-8693;
92. Wilde, A., Fiedler, B., and Bonier, T. (2002). The cyanobacterial phytochrome Cph2 inhibits phototaxis towards blue light. Mol. Microbiol. 44, 981-988;
93. Wilde, A., Churin, Y., Schubert, H., and Borner, T. (1997). Disruption of a Synechocystis sp. PCC 6803 gene with partial similarity to phytochrome genes alters growth under changing light qualities. FEBS Lett. 406, 89-92;
94. Yeh, K. C., Wu, S.-H., Murphy, J. T., and Lagarias, J. C. (1997). A cyanobacterial phytochrome two-component light sensory system. Science 277, 1505-1508;
95. Yoshihara, S., Suzuki, F., Fujita, H., Geng, X. X., and Ikeuchi, M. (2000). Novel putative photoreceptor and regulatory genes required for the positive phototactic movement of the unicellular motile cyanobacterium Synechocystis sp. PCC 6803. Plant Cell Physiol. 41, 1299-1304;
96. Kehoe, D. M. and A. Gutu. 2006. Annu Rev Plant Biol 57:127-150;
97. Park, H., Saha, S. K. and M. Inouye. 1998. Proc Natl Acad Sci USA 95: 6728-6732; and
98. Levchenko, I., Seidel, M., Sauer, R. T. and T. A. Baker. 2000. Science 289: 2354-2356.

What is claimed is:

1. A process for regulating transcriptional activity of a gene, comprising:
introducing genetic material into an organism, the genetic material being selected from a photosynthetic bacterium;
placing the gene downstream of a light-regulated promoter within the organism, the light-regulated promoter having a light responsive element; and
subjecting the light-regulated promoter to an exogenous light source, the light source being configured to control the level of transcriptional activity of the gene.

2. The process of claim 1, wherein the gene is selected from at least one of a prokaryotic gene and a eukaryotic gene.

3. The process of claim 1, wherein the organism is a prokaryotic organism.

4. The process of claim 1, wherein the photosynthetic bacterium comprises a cyanobacterium.

5. The process of claim 1, wherein the genetic material is from a cyanobacterial photoreceptor.

6. The process of claim 5, wherein the cyanobacterial photoreceptor comprises genes selected from at least one of ho1, pixJ1, cph1, pcyA, rcaE, rcaF and rcaC.

7. The process of claim 6, wherein the pixJ1 and cph1 genes are both obtained from a cyanobacterium of the genera Synechocystis.

8. The process of claim 5, wherein the photoreceptor includes at least one of an RcaE photoreceptor and a fusion photoreceptor, the fusion photoreceptor being selected from at least one of PixJ1/RcaE and Cph1/RcaE.

9. The process of claim 1, wherein the light-regulated promoter comprises a cpc2 promoter obtained from at least one of Synechococcus sp. PCC7335 and Fremyella diplosiphon.

10. The process of claim 1, wherein the light source comprises at least one of red/green light, blue/green light and red/far red light.

11. The process of claim 1, wherein the transcriptional activity of the gene is controlled by adjusting the ratio of light colors provided by the light source.

12. A process for regulating transcriptional activity of a gene, comprising:
introducing genetic material from a cyanobacterial photoreceptor into a prokaryotic organism;
placing a gene in the organism downstream of a cpc2 promoter, the cpc2 promoter having a light responsive element; and
subjecting the organism to an exogenous light source, the light source being configured to control the level of transcriptional activity in the gene by adjusting the ratio of light colors provided by the light source;
wherein the cpc2 promoter is obtained from at least one of Synechococcus sp. PCC7335 and Fremyella diplosiphon.

13. The process of claim 12, wherein the gene is selected from at least one of a prokaryotic gene and a eukaryotic gene.

14. The process of claim 12, wherein the prokaryotic organism is Escherichia coli.

15. The process of claim 12, wherein the cyanobacterial photoreceptor comprises genes selected at least one of ho1, pixJ1, cph1, pcyA, rcaE, rcaF and rcaC, the pixJ1 and cph1 genes both being obtained from a cyanobacterium of the genera Synechocystis.

16. The process of claim 12, wherein the light source comprises at least one of red/green light, blue/green light and red/far red light.

17. The process of claim 12, wherein the photoreceptor includes at least one of an RcaE photoreceptor and a fusion photoreceptor, the fusion photoreceptor being selected from at least one of PixJ1/RcaE and Cph1/RcaE.

18. A process for regulating transcriptional activity in Escherichia coli, comprising:
introducing genetic material from a cyanobacteria) photoreceptor into the Escherichia coli, the photoreceptor including genes selected from at least one of ho1, pixJ1, cph1, pcyA, rcaE, rcaF and rcaC;
placing a gene in the organism downstream of a cpc2 promoter, the cpc2 promoter having a light responsive element and being obtained from at least one of Synechococcus sp. PCC7335 and Fremyella diplosiphon; and
subjecting the organism to an exogenous light source, the light source being configured to control the level of transcriptional activity in the gene by adjusting the ratio of light colors provided by the light source.

19. The process of claim 18, wherein the gene is selected from at least one of a prokaryotic gene and a eukaryotic gene and the light source comprises at least one of red/green light, blue/green light and red/far red light.

20. The process of claim 18, wherein the pixJ1 and cph1 genes both are obtained from a cyanobacterium of the genera Synechocystis.
